(19) United States
(12) Patent Application Publication
(10) Pub. No.: US 2026/0209803 A1
(43) Pub. Date: Jul. 23, 2026

(54) COMPOSITIONS AND METHODS FOR MODIFYING A TARGET NUCLEIC ACID

(71) Applicant: The Regents of the University of California, Oakland, CA (US)

(72) Inventors: Atip Lawanprasert, Oakland, CA (US); Joy Chen, Oakland, CA (US); Niren Murthy, El Cerrito, CA (US); David N. Nguyen, Oakland, CA (US); Matthew Kan, Oakland, CA (US); Kai Chen, Berkeley, CA (US); Jennifer A. Doudna, Berkeley, CA (US)

(21) Appl. No.: 19/440,302

(22) Filed: Jan. 5, 2026

Related U.S. Application Data
(60) Provisional application No. 63/742,127, filed on Jan. 6, 2025.

(51) Int. Cl. C12N 15/90; C12N 9/22; C12N 15/11; C12N 15/88

(52) U.S. Cl. CPC C12N 15/907; C12N 9/226; C12N 15/11; C12N 15/88; C12N 2310/20

(57) ABSTRACT

The disclosure provides compositions and methods for modifying a target nucleic acid. In some embodiments, a composition can include a lipid nanoparticle including a targetable nuclease or an mRNA encoding a targetable nuclease, a DNA-binding protein or an mRNA encoding a DNA-binding protein, and a donor template comprising a homology directed repair (HDR) template and one or more DNA-binding protein target sequences. In some embodiments, a composition can include a Cas protein, one or more single guide RNAs (sgRNAs), and a lipid nanoparticle.

### CROSS-REFERENCE

This application claims the benefit of U.S. Provisional Patent Application No. 63/742,127 filed Jan. 6, 2025, which application is incorporated herein by reference in its entirety.

### STATEMENT REGARDING FEDERALLY SPONSORED RESEARCH

This invention was made with government support under Grant Number AI153767 awarded by the National Institutes of Health and under Grant Number 2146752 awarded by the National Science Foundation. The government has certain rights in the invention.

### INCORPORATION BY REFERENCE OF SEQUENCE LISTING PROVIDED AS AN XML FILE

A Sequence Listing is provided herewith as a Sequence Listing XML, “UCSF-826WO_SEQ_LISTING.xml” created on Dec. 16, 2025 and having a size of 17,404 bytes. The contents of the Sequence Listing XML are incorporated by reference herein in their entirety.

### I. INTRODUCTION

The application of clustered regularly interspaced short palindromic repeats (CRISPR) and CRISPR-associated (Cas) proteins has revolutionized molecular biology by making genome editing possible. CRISPR-mediated gene editing is a powerful and practical tool with potential for creating new scientific tools, correcting clinically relevant mutations, and engineering new cell-based immunotherapies. Viral delivery vectors and electroporation have emerged as two strategies for CRISPR-based editing in immune cells. However, the field of gene therapy has been fraught with complications of viral delivery vectors, including size limitations, the possibility of an inflammatory or immune response, lack of efficacy, and high cost and low production levels of viral vectors.

Non-viral strategies to deliver the CRISPR-Cas system into the cell for genetic modification, such as electroporation, avoid many complications associated with viral delivery, such as fatal systemic immune responses to viral vectors, viral delivery inefficiency, and viral insertion-related gene overexpression. However, in some cases, poor stability, RNP complex aggregation into micron-sized particles, the need for large amounts of HDR template, and dose-dependent cytotoxicity can occur when non-viral strategies for CRISPR-Cas system delivery are employed. For example, Cas9 proteins can be unstable when complexed with the single-guide RNA (sgRNA), forming cloudy precipitates immediately or over a few hours of time, which correlate with reduced editing efficiency.

While CRISPR-Cas9 targeted insertion of novel sequences enabled by homology-directed repair (HDR) offers tremendous engineering and translational opportunities, a major barrier is the inability to efficiently deliver both the editing enzyme and the DNA repair template into the nucleus, where HDR occurs.

There is a need for compositions and methods that provide efficient gene editing, and such is provided herein.

### II. SUMMARY

As demonstrated in the working examples below, the inventors have shown that lipid nanoparticles can be used to effectively deliver gene editing components, such as CRISPR-Cas effector proteins (or mRNAs encoding them), gRNAs, and HDR templates, to the nucleus of a cell.

Provided here are methods and compositions for delivering gene editing components for modifying a target nucleic acid.

In one aspect, the disclosure features a composition for modifying a target nucleic acid, comprising a lipid nanoparticle (LNP) that comprises (a) an mRNA encoding a targetable nuclease; (b) an mRNA encoding a DNA-binding protein; and (c) a donor template comprising: (i) a homology directed repair (HDR) template comprising a nucleic acid sequence for insertion into the target nucleic acid; and (ii) one or more DNA-binding protein target sequences. In some cases, the donor template comprises one or more DNA-binding protein target sequences in the region 5′ of the HDR template. In some cases, the donor template comprises one or more DNA-binding protein target sequences in the region 3′ of the HDR template. In some cases, the donor template comprises one or more DNA-binding protein target sequences in the region 5′ of the HDR template and one or more DNA-binding protein target sequences in the region 3′ of the HDR template.

In some embodiments of this aspect, the LNP comprises one or more ionizable lipids. In some cases, the LNP comprises OF-02, DOPE, cholesterol, DMG-PEG 2000, or DOTAP, or any combination thereof. In other cases, the LNP comprises: L-319, DOPE, cholesterol, or DMG-PEG 2000, or any combination thereof.

In some embodiments, the targetable nuclease is a first RNA-guided nuclease and the DNA-binding protein is a second RNA-guided nuclease. In some cases, one RNA-guided nuclease is both the targetable nuclease and the DNA-binding protein. In some cases, the RNA-guided nuclease is a CRISPR-Cas effector protein.

In some embodiments, where the composition comprises a RNA-guided nuclease, the composition further comprises a target guide RNA (gRNA) and a donor gRNA; wherein the donor template further comprises one or more protospacer adjacent motifs (PAMs); wherein the target gRNA is complementary to the target nucleic acid; and wherein at least one of said one or more DNA-binding protein target sequences hybridizes to the donor gRNA's guide sequence or a portion thereof. In some cases, one guide RNA is both the target gRNA and the donor gRNA. In some cases, the target gRNA and the donor gRNA have different sequences.

In some embodiments, at least one of the one or more DNA-binding protein target sequences of the donor template is a truncated CRISPR-Cas targeting sequence (tCTS), and a PAM is adjacent to each tCTS. In some cases, the donor template comprises two tCTSs and two PAMs. In some cases, the donor template includes more than two tCTSs (e.g., 3, 4, 5, 6, 7, 8, 9, or 10 tCTSs).

In some embodiments, the DNA-binding protein comprises a transcription activator-like effector (TALE) DNA-binding protein or a zinc finger (ZF) DNA-binding protein.

In another aspect, the disclosure features a composition for modifying a target nucleic acid, comprising a lipid nanoparticle (LNP) that comprises (a) an ionizable lipid selected from OF-02 or L-319; (b) a targetable nuclease or an mRNA encoding the targetable nuclease; (c) a DNA-binding protein or an mRNA encoding the DNA-binding protein; and (d) a donor template comprising: (i) a homology directed repair (HDR) template comprising a nucleic acid sequence for insertion into the target nucleic acid; and (ii) one or more DNA-binding protein target sequences. In some cases, the donor template comprises one or more DNA-binding protein target sequences in the region 5′ of the HDR template. In some cases, the donor template comprises one or more DNA-binding protein target sequences in the region 3′ of the HDR template. In some cases, the donor template comprises one or more DNA-binding protein target sequences in the region 5′ of the HDR template and one or more DNA-binding protein target sequences in the region 3′ of the HDR template.

In some embodiments of this aspect, the LNP comprises: OF-02, L-319, DOPE, cholesterol, DMG-PEG 2000, or DOTAP, or any combination thereof. In some cases, the LNP comprises 15 mol % to 25 mol % OF-02, 20 mol % to 30 mol % DOPE, 35 mol % to 45 mol % cholesterol, 0.5 mol % to 1.5 mol % DMG-PEG 2000, and 15 mol % to 25 mol % DOTAP of the total lipids. In some cases, the LNP comprises 30 mol % to 40 mol % L-319, 20 mol % to 30 mol % DOPE, 35 mol % to 45 mol % cholesterol, and 0.5 mol % to 1.5 mol % DMG-PEG 2000 of the total lipids.

In some embodiments, (a) each of the targetable nuclease and the DNA-binding protein is an RNA-guided nuclease; (b) the composition comprises a target guide RNA (gRNA) and a donor gRNA; (c) the donor template comprises one or more protospacer adjacent motifs (PAMs); (d) the target gRNA is complementary to the target nucleic acid.

In some embodiments, one RNA-guided nuclease (e.g., a CRISPR-Cas protein such as Cas9) is both the targetable nuclease and the DNA-binding protein. In some cases, the RNA-guided nuclease is a CRISPR-Cas effector protein. In some cases, one guide RNA is both the target gRNA and the donor gRNA (i.e., the one guide RNA serves as both the target gRNA and the donor gRNA). In some cases, the one or more DNA-binding protein target sequences of the donor template are truncated CRISPR-Cas targeting sequences (tCTS) that hybridize to the donor gRNA's guide sequence or a portion thereof, and a PAM is adjacent to each tCTS. In some cases, the donor template comprises two tCTSs and two PAMs. In some cases, the donor template includes more than two tCTSs (e.g., 3, 4, 5, 6, 7, 8, 9, or 10 tCTSs).

In some embodiments, where the composition comprises a RNA-guided nuclease that is a CRISPR-Cas effector protein, the CRISPR-Cas effector protein is complexed with the target gRNA or the donor gRNA as a ribonucleoprotein (RNP). In some cases, the RNP comprising the donor gRNA is complexed with the donor template.

In some embodiments, the DNA-binding protein comprises a transcription activator-like effector (TALE) DNA-binding protein or a zinc finger (ZF) DNA-binding protein.

In another aspect, the disclosure features a method for modifying a target nucleic acid, comprising contacting a cell with any of the compositions described above, wherein the nucleic acid sequence for insertion is integrated into the target nucleic acid.

In some embodiments, the method is performed in vivo, in vitro, or ex vivo. In some embodiments, the method is performed on a human cell. In some cases, the cell is a primary cell. In some cases, the cell is a T cell. In some cases, the nucleic acid sequence for insertion is integrated into the target nucleic acid at a T-cell receptor a constant (TRAC) locus. In some cases, the HDR template of the donor template encodes a chimeric antigen receptor (CAR) for insertion.

Also provided in the disclosure are reagents, compositions, and kits/systems that find use in practicing the subject methods.

### III. BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1 depicts a schematic representation of LNP-mediated delivery for efficient HDR in human T-cells. Although the figure depicts addition of tCTS via PCR, a tCTS can be added to a donor template using any convenient method, e.g., using any convenient molecular biology technique (e.g., cloned directly into a plasmid).

FIG. 2 provides results for LNP-mediated large gene (Rab11-sfGFP) knock-in to the Rab11 locus in primary human T-cells.

FIG. 3A-3C provides results demonstrating that truncated Cas9-targeted sequences (tCTS) improve LNP-mediated HDR knock-in efficiency in primary human T cells after 3, 5, and 7 days post-transfection at a dose of 50 ug/million cells.

FIG. 4A-4C provides additional results demonstrating that truncated Cas9-targeted sequences (tCTS) improve HDR knock-in efficiency in primary human T cells after 3 days post-transfection.

FIG. 5A-5F provides results demonstrating that truncated Cas9-targeted sequences (tCTS) improve LNP-mediated HDR knock-in efficiency in primary human T cells after 3, 5, and 7 days post-transfection at a dose of 10 ug/million cells.

FIG. 6A-6B provides the results of LNP-mediated HDR knock-in to HEK293T cells with donor DNA-sgRNA-Cas9 ribonucleoprotein (RNP) complex. HEK293 cells were treated with an equivalence of 50 pmol and 100 pmol.

FIG. 7A-7B provides the results of LNP-mediated HDR knock-in to HEK293T cells with donor DNA-sgRNA-Cas9 ribonucleoprotein (RNP) complex with or without tCTS and with or without polyglutamic acid (PGA).

FIG. 8A-8B demonstrates that lipid nanoparticle delivery of Cas9 RNP to HEK293T cells using a donor template with tCTSs (e.g., delivery of ssHRTc mNeon with Cas9 RNP to HEK293T cells) resulted in durable targeted integration in AAVS1. A) Representative flow cytometry plot of mNeon in HEK293T cells 20 days after lipid nanoparticle administration. Quantifications are shown on the right. B) ddPCR copy number analysis by in-out PCR for knock-in at AAVS1 compared to mNeon expression.

### IV. DEFINITIONS

As used herein, the “CRISPR-Cas” system refers to a class of bacterial systems for defense against foreign nucleic acid. CRISPR-Cas systems are found in a wide range of eubacterial and archaeal organisms. CRISPR-Cas systems include type I, II, and III sub-types. Wild-type type II CRISPR-Cas systems utilize an RNA-mediated nuclease, for example, Cas9 protein, in complex with guide and activating RNA (e.g., single-guide RNA or sgRNA) to recognize and cleave foreign nucleic acids, i.e., foreign nucleic acids including natural or modified nucleotides.

As used herein, the term “targetable nuclease” refers to a protein that can recognize a sequence of a target nucleic acid (e.g., a target gene within a genome) and bind to the target nucleic acid. In some embodiments, the targetable nuclease can modify the target nucleic acid. In some embodiments, a targetable nuclease can be an RNA-guided nuclease, e.g., a Cas protein. In other embodiments, a targetable nuclease can be a fusion protein that includes a protein that can bind to the target nucleic acid (e.g., a transcription activator-like (TAL) effector DNA-binding protein or a zinc finger DNA-binding protein) and a protein that can modify the target nucleic acid (e.g., a nuclease, a transcription activator or repressor). In some embodiments, the targetable nuclease has nuclease activity. In other embodiments, the targetable nuclease does not have nuclease activity. In some embodiments, the targetable nuclease can modify the target nucleic acid by cleaving the target nucleic acid. The cleaved target nucleic acid can then undergo homologous recombination with a nearby a homology directed repair (HDR) template. In other embodiments, the targetable nuclease (e.g., a targetable nuclease without any nuclease activity) can regulate the expression of the target nucleic acid. For example, a targetable nuclease can be a fusion protein containing a TAL effector DNA-binding protein and a transcription activator.

As used herein, the term “DNA-binding protein” refers to a protein that can directly or indirectly bind to a DNA-binding protein target sequence within a donor template (which includes an HDR template). Without being bound by any theory, the DNA-binding protein serves to transport or shuttle the donor template to a cellular location close to the target nucleic acid. Thus, the DNA-binding protein can improve the delivery of the HDR template into target cells, especially to the cell nucleus, and increase knock-in efficiencies. In some embodiments, the DNA-binding protein can be a transcription activator-like (TAL) effector DNA-binding protein or a zinc finger DNA-binding protein. Each of the transcription activator-like (TAL) effector DNA-binding protein and zinc finger DNA-binding protein can directly bind to a DNA-binding protein target sequence within a donor template. In some embodiments, the DNA-binding protein can be an RNA-guided nuclease, e.g., a Cas protein, which can indirectly bind to a DNA-binding protein target sequence within a donor template via a donor gRNA.

As used herein, the term “donor template” refers a polynucleotide that includes a homology directed repair (HDR) template and one or more DNA-binding protein target sequences. An HDR template can include a 5′ homology arm, a nucleotide insert (e.g., an exogenous sequence and/or a sequence that encodes a heterologous protein or fragment thereof), and a 3′ homology arm. In some embodiments, the donor template can also include one or more edge sequences at one or both termini of the donor template. As described further herein, pre-incubation of the RNP complex containing the DNA-binding protein (e.g., a Cas protein) and donor gRNA and the donor template prior to electroporation improves in knock-in efficiency.

As used herein, the term “DNA-binding protein target sequence” (also referred to as “DNA-binding protein targeting sequence”) refers to a nucleotide sequence that is recognized and bound by a DNA-binding protein. In some embodiments, the DNA-binding protein, e.g., a transcription activator-like (TAL) effector DNA-binding protein or zinc finger DNA-binding protein, can directly recognize and bind a DNA-binding protein target sequence. In other embodiments, a DNA-binding protein, e.g., an RNA-guided nuclease, can indirectly recognize and bind a DNA-binding protein target sequence via a donor gRNA. The DNA-binding protein, e.g., the RNA-guided nuclease, binds to the donor gRNA, which hybridizes to the DNA-binding protein target sequence. In some embodiments, the DNA-binding protein target sequence has the same sequence as a portion of the target nucleic acid.

In some embodiments, the DNA-binding protein target sequence hybridizes to a portion of the guide sequence of the donor gRNA, e.g., the DNA-binding protein target sequence is truncated or includes mismatches compared to the guide sequence of the donor gRNA. In some cases, where the DNA-binding protein target sequence has the same sequence as a portion of the target nucleic acid and hybridizes to a portion of the guide sequence of the donor gRNA, the DNA-binding protein target sequence is referred to as a “truncated CRISPR-Cas targeting sequence” (“tCTS”). As an illustrative example, if a guide sequence of a guide RNA is 20 nt, the target sequence of the target DNA is also 20 nt, but the tCTS is shorter (e.g., 10-16 nt) such that a CRISPR-Cas effector protein (e.g., Cas9) can be guided to and will associate with the tCTS without cleaving it. For more information related to tCTS, refer to Nguyen et al., Nat Biotechnol. 2020 January; 38(1):44-49; Nguyen et al., (2019) bioRxiv 591719; doi: https://doi.org/10.1101/591719; and to US Patent Application Publication US20220017882, all of which are incorporated herein by reference.

As used herein, the “RNA-guided nuclease” refers to a nuclease that binds to a guide RNA (gRNA) and utilizes the gRNA to search for regions within a DNA polynucleotide that it can target. In general, an RNA-guided nuclease can target nearly any sequence within the DNA polynucleotide that is complementary to the gRNA. In some embodiments, the RNA-guided nuclease has nuclease activity and can cleave the linkage (e.g., phosphodiester bonds) between nucleotides in the DNA polynucleotide. In other embodiments, the RNA-guided nuclease does not have nuclease activity and can be used to target or localize other proteins (e.g., transcriptional activator or repressors) that are fused to the RNA-guided nuclease to the region of interest within the DNA polynucleotide.

As used herein, the term “guide RNA” or “gRNA” refers to a DNA-targeting RNA that can guide an RNA-guided nuclease (e.g., a Cas protein) to a target nucleic acid by hybridizing to the target nucleic acid. In some embodiments, a guide RNA can be a single-guide RNA (sgRNA), which contains a guide sequence (i.e., crRNA equivalent portion of the single-guide RNA) that targets the RNA-guided nuclease to the target nucleic acid and a scaffold sequence (i.e., tracrRNA equivalent portion of the single-guide RNA) that interacts with the RNA-guided nuclease. In other embodiments, a guide RNA can contain two components, a guide sequence (i.e., crRNA equivalent portion of the single-guide RNA) that targets the RNA-guided nuclease to the target nucleic acid and a scaffold sequence (i.e., tracrRNA equivalent portion of the single-guide RNA) that interacts with the RNA-guided nuclease. A portion of the guide sequence can hybridize to a portion of the scaffold sequence to form the two-component guide RNA.

As used herein, the term “target guide RNA” or “target gRNA” refers to a gRNA that can hybridize to the target nucleic acid, e.g., at a location in the target nucleic acid where integration of the HDR template happens.

As used herein, the term “donor guide RNA” or “donor gRNA” refers to a gRNA that can hybridize a DNA-binding protein target sequence within a donor template. In some embodiments, a DNA-binding protein target sequence can be complementary (e.g., partially complementary or completely complementary) to an equal length portion of the sequence of a donor gRNA.

As used herein, the term “single-guide RNA” or “sgRNA” refers to a DNA-targeting RNA containing a guide sequence (i.e., crRNA equivalent portion of the single-guide RNA) that targets the Cas protein to the target DNA and a scaffold sequence (i.e., tracrRNA equivalent portion of the single-guide RNA) that interacts with the Cas protein.

As used herein, the term “complementary” or “complementarity” refers to the capacity for base pairing between nucleobases, nucleosides, or nucleotides, as well as the capacity for base pairing between one polynucleotide to another polynucleotide. In some embodiments, one polynucleotide can have “complete complementarity,” or be “completely complementary,” to another polynucleotide, which means that when the two polynucleotides are optionally aligned, each nucleotide in one polynucleotide can engage in Watson-Crick base pairing with its corresponding nucleotide in the other polynucleotide. In other embodiments, one polynucleotide can have “partial complementarity,” or be “partially complementary,” to another polynucleotide, which means that when the two polynucleotides are optionally aligned, at least 60% (e.g., 65%, 70%, 75%, 80%, 85%, 90%, 95%, or 97%) but less than 100% of the nucleotides in one polynucleotide can engage in Watson-Crick base pairing with their corresponding nucleotides in the other polynucleotide. In other words, there is at least one (e.g., one, two, three, four, five, six, seven, eight, nine, or ten) mismatched nucleotide base pair when the two polynucleotides are hybridized. Pairs of nucleotides that engage in Watson-Crick base pairing includes, e.g., adenine and thymine, cytosine and guanine, and adenine and uracil, which all pair through the formation of hydrogen bonds. Examples of mismatched bases include a guanine and uracil, guanine and thymine, and adenine and cytosine pairing.

As used herein, the term “Cas protein”, also referred to herein as a “CRISPR-Cas effector protein” refers to a Clustered Regularly Interspaced Short Palindromic Repeats-associated protein that has effector function, i.e., in nature the protein acts as a nuclease to cleave a target nucleic acid (DNA or RNA). A Cas protein can be a wild-type Cas protein or a Cas protein variant. Cas9 protein is an example of a Cas protein that belongs in the type II CRISPR-Cas system (e.g., Rath et al., Biochimie 117:119, 2015). Other examples of Cas proteins are discussed further herein. In some cases, a naturally-occurring Cas protein (e.g., Cas9) uses both a crRNA and a tracrRNA for site-specific DNA recognition and cleavage. The crRNA associates, through a region of partial complementarity, with the tracrRNA to guide the Cas protein to a region homologous to the crRNA in the target DNA called a “protospacer”. A naturally-occurring Cas protein (e.g., Cas9, Cas12a, etc.) cleaves DNA at sites specified by a guide sequence contained within a crRNA. In some embodiments of the compositions and methods described herein, a Cas protein associates with a target gRNA or a donor gRNA to form a ribonucleoprotein (RNP) complex. In some embodiments of the compositions and methods described herein, the Cas protein has nuclease activity. In other embodiments, the Cas protein does not have nuclease activity.

As used herein, the term “Cas protein variant” refers to a Cas protein that has at least one amino acid substitution (e.g., one, two, three, four, five, six, seven, eight, nine, ten, or more amino acid substitutions) relative to the sequence of a wild-type Cas protein and/or is a truncated version or fragment of a wild-type Cas protein. In some embodiments, a Cas protein variant has at least 75% sequence identity (e.g., at least 75%, 80%, 85%, 90%, 91%, 92%, 93%, 94% 95%, 96%, 97%, 98%, 99%, or 100% sequence identity) to the sequence of a wild-type Cas protein. In some embodiments, a Cas protein variant is a fragment of a wild-type Cas protein and has at least one amino acid substitution relative to the sequence of the wild-type Cas protein. A Cas protein variant can be a Cas9 protein variant. In some embodiments, a Cas protein variant has nuclease activity. In other embodiments, a Cas protein variant does not have nuclease activity.

As used herein, the term “ribonucleoprotein complex” or “RNP complex” refers to a complex comprising a Cas protein or variant (e.g., a Cas9 protein or variant) and a gRNA.

As used herein, the term “modifying” in the context of modifying a target nucleic acid in the genome of a cell refers to inducing a change (e.g., cleavage) in the target nucleic acid. In some embodiments, the change can be a structural change in the sequence of the target nucleic acid. For example, the modifying can take the form of inserting a nucleotide sequence into the target nucleic acid. For example, an exogenous nucleotide sequence can be inserted into the target nucleic acid. The target nucleic acid can also be excised and replaced with an exogenous nucleotide sequence. In another example, the modifying can take the form of cleaving the target nucleic acid without inserting a nucleotide sequence into the target nucleic acid. For example, the target nucleic acid can be cleaved and excised. Such modifying can be performed, for example, by inducing a double stranded break within the target nucleic acid, or a pair of single stranded nicks on opposite strands and flanking the target nucleic acid. Methods for inducing single or double stranded breaks at or within a target nucleic acid include the use of a targetable nuclease (e.g., a Cas protein) as described herein directed to the target nucleic acid. In other embodiments, modifying a target nucleic acid includes targeting another protein to the target nucleic acid and does not include cleaving the target nucleic acid.

As used herein, the phrase “lipid nanoparticle” (LNP) refers to a transfer vehicle comprising one or more lipids (e.g., ionizable lipids, cationic lipids, non-cationic lipids, neutral lipids, neutral phospholipids, polymerizable lipids, PEG-modified lipids, cholesterol, and the like). In some cases, an LNP includes a molecular payload.

As used herein, the term “anionic polymer” refers to a molecule composed of multiple subunits or monomers that has an overall negative charge. Each subunit or monomer in a polymer can, independently, be an amino acid, a small organic molecule (e.g., an organic acid), a sugar molecule (e.g., a monosaccharide or a disaccharide), or a nucleotide. An anionic polymer can contain multiple amino acids, small organic molecules (e.g., organic acids), nucleotides (e.g., natural or non-natural nucleotides, or analogues thereof), or a combination thereof. An anionic polymer can be an anionic homopolymer where all subunits or monomers in the polymer are the same. An anionic polymer can be an anionic heteropolymer where the subunits and monomers in the polymer are different. An anionic polymer does not refer to a nucleic acid, such as a deoxyribonucleic acid (DNA), ribonucleic acid (RNA), that is composed entirely of nucleotides. However, an anionic polymer can include one or more nucleobases (e.g., guanosine, cytidine, adenosine, thymidine, and uridine) together with other subunits or monomers, such as amino acids and/or small organic molecules (e.g., an organic acid). In some embodiments, at least 50% (e.g., 55%, 60%, 65%, 70%, 75%, 80%, 85%, 90%, 95%, or 100%) of the subunits or monomers in the polymer are not nucleotides or do not contain nucleobases. An anionic polymer can be an anionic polypeptide or an anionic polysaccharide. An anionic polymer can contain at least two subunits or monomers (e.g., at least 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400 subunits or monomers; between 100 and 400, between 120 and 400, between 140 and 400, between 160 and 400, between 180 and 400, between 200 and 400, between 220 and 400, between 240 and 400, between 260 and 400, between 280 and 400, between 300 and 400, between 320 and 400, between 340 and 400, between 360 and 400, between 380 and 400, between 100 and 380, between 100 and 360, between 100 and 340, between 100 and 320, between 100 and 300, between 100 and 280, between 100 and 260, between 100 and 240, between 100 and 220, between 100 and 200, between 100 and 180, between 100 and 160, between 100 and 140, or between 100 and 120 subunits or monomers).

As used herein, the term “anionic polypeptide” refers to an anionic polymer that has at least 50% (e.g., 55%, 60%, 65%, 70%, 75%, 80%, 85%, 90%, 95%, or 100%) of its subunits or monomers being amino acids, such as acidic amino acids (e.g., glutamic acids and aspartic acids), or derivatives thereof. Aside from amino acids, an anionic polypeptide can also contain small organic molecules (e.g., organic acids), sugar molecules (e.g., monosaccharides or disaccharides), or nucleotides. In some embodiments, an anionic polypeptide can be a homopolymer where all of its subunits are the same. In other embodiments, an anionic polypeptide can be a heteropolymer that contains two or more different subunits. For example, an anionic polypeptide can be polyglutamic acid (PGA) (e.g., poly-gamma-glutamic acid), polyaspartic acid, and polycarboxyglutamic acid. In another example, an anionic polypeptide can contain a mixture of glutamic acids and aspartic acids. In some embodiments, at least 50% (e.g., 55%, 60%, 65%, 70%, 75%, 80%, 85%, 90%, 95%, or 100%) of the subunits or monomers in an anionic polypeptide can be glutamic acids and/or aspartic acids. An anionic polypeptide can contain at least two subunits or monomers (e.g., at least 5, 10, 15, 20, 25, 30, 35, 40, 45, 50, 55, 60, 65, 70, 75, 80, 85, 90, 95, 100, 110, 120, 130, 140, 150, 160, 170,180, 190, 200, 210, 220, 230, 240, 250, 260, 270, 280, 290, 300, 310, 320, 330, 340, 350, 360, 370, 380, 390, or 400 subunits or monomers; between 100 and 400, between 120 and 400, between 140 and 400, between 160 and 400, between 180 and 400, between 200 and 400, between 220 and 400, between 240 and 400, between 260 and 400, between 280 and 400, between 300 and 400, between 320 and 400, between 340 and 400, between 360 and 400, between 380 and 400, between 100 and 380, between 100 and 360, between 100 and 340, between 100 and 320, between 100 and 300, between 100 and 280, between 100 and 260, between 100 and 240, between 100 and 220, between 100 and 200, between 100 and 180, between 100 and 160, between 100 and 140, or between 100 and 120 subunits or monomers).

As used herein, the term “active for at least one week” refers to a Cas protein in the presence of a lipid nanoparticle, or a Cas protein and sgRNA RNP complex formed in the presence of a lipid nanoparticle, that has activity (e.g., nuclease activity) for at least one week (e.g., at least 2, 3, 4, 5, 6, 7, 8, 9, 10, 11, 12, 13, 14, 15, 16, 17, 18, 19, 20, 21, 22, 23, or 24 weeks) in liquid form at room temperature, 4° C., or 37° C. In the case where the composition comprising the Cas protein and the lipid nanoparticle or the composition comprising the Cas protein, the lipid nanoparticle, and the sgRNA is a lyophilized composition, the term “active for at least one week” also refers to that the Cas protein is active for at least one week at room temperature, 4° C., or 37° C. after reconstituting the lyophilized composition into liquid form. In other words, in some embodiments, if the composition containing the Cas protein and the lipid nanoparticle or the composition containing the Cas protein and sgRNA RNP complexes and the lipid nanoparticle is lyophilized and later reconstituted, the Cas protein has activity for at least one week (e.g., at least 2, 3, 4, 5, 6, 7, 8, 9, 10, 11, 12, 13, 14, 15, 16, 17, 18, 19, 20, 21, 22, 23, or 24 weeks) at room temperature, 4° C., or 37° C. after reconstitution.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. As such, the articles “a” and “an” are used herein to refer to one or to more than one (i.e., to at least one) of the grammatical object of the article. By way of example, “an element” means one element or more than one element. Thus, for example, reference to “a cell” includes a plurality of such cells and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. For example, it is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### V. DETAILED DESCRIPTION

The disclosure provides compositions and methods for modifying a target nucleic acid that include: (a) a targetable nuclease or mRNA encoding a targetable nuclease; (b) a DNA-binding protein or mRNA encoding a DNA-binding protein; and (c) a donor template comprising a homology directed repair (HDR) template and one or more DNA-binding protein target sequences. The DNA-binding protein can directly or indirectly bind to the DNA-binding protein target sequence within the donor template. As described in detail further herein, in some embodiments, when the DNA-binding protein is a transcription activator-like (TAL) effector DNA-binding protein, the TAL effector DNA-binding protein can directly recognize and bind to the DNA-binding target sequence. In some embodiments, when the DNA-binding protein is a zinc finger DNA-binding protein, the zinc finger DNA-binding protein can directly recognize and bind to the DNA-binding target sequence. In other embodiments, when the DNA-binding protein is an RNA-guided nuclease (e.g., a Cas protein such as Cas9), the RNA-guided nuclease can indirectly bind to a DNA-binding protein target sequence via a donor gRNA, which can hybridize to the DNA-binding protein target sequence. Without being bound by any theory, the DNA-binding protein serves to transport or shuttle the donor template to a cellular location, e.g., the nucleus. Thus, the DNA-binding protein can improve the delivery of the HDR template into target cells, especially to the cell nucleus, and increase knock-in efficiencies.

The disclosure provides compositions and methods for modifying a target nucleic acid that include a Cas protein (e.g., a Cas9 protein), one or more single guide RNAs (sgRNAs), and a lipid nanoparticle. Without being bound by any theory, the addition of a lipid nanoparticle to the Cas protein and sgRNA ribonucleoprotein (RNP) complex stabilizes the complex and prevents aggregation.

### CRISPR-Cas

In some embodiments of the compositions and methods described herein, the targetable nuclease is a first RNA-guided nuclease, the DNA-binding protein is a second RNA-guided nuclease, and the donor template further comprises one or more protospacer adjacent motifs (PAMs). In some cases, one or more of the RNA-guided nucleases is provided as an mRNA encoding the RNA-guided nuclease. The composition also further comprises a target guide RNA (gRNA) that is complementary to the target nucleic acid and a donor gRNA that hybridizes to the DNA-binding protein target sequence. The target gRNA can form a first RNP complex with the first RNA-guided nuclease and guide the first RNA-guided nuclease (e.g., Cas protein such as Cas9) to the target nucleic acid. In some embodiments, the guide sequence of the target gRNA (e.g., a guide sequence that is at least 15 nucleotides (e.g., 15, 16, 17, 18, 19, 20, 21, 22, 23, 24, or 25 nucleotides) is complementary to the target nucleic acid. The donor gRNA can form a second RNP with the second RNA-guided nuclease. The DNA-binding protein target sequence in the donor template can hybridize to the guide sequence of the donor gRNA or a portion of the guide sequence of the donor gRNA. Therefore, the complex containing the second RNA-guided nuclease, the donor gRNA, and the donor template can bring the donor template into the desired intracellular location (e.g., the nucleus) for homologous recombination to occur at the integration site in the target nucleic acid. In some embodiments, the sequences of the target gRNA and the donor gRNA are the same. In some embodiments, the sequences of the target gRNA and the donor gRNA are different. In some embodiments, the first and second RNA-guided nucleases are the same. In other embodiments, the first and second RNA-guided nucleases are different. In other embodiments, a composition described herein can also be used for integrating the donor template through non-homology directed repair mediated methods, such as homology independent targeted integration (HITI).

The composition can contain the targetable nuclease and the target gRNA in a molar ratio of between 1:10 and 2:1 (e.g., between 1:5 and 2:1, between 2:5 and 2:1, between 3:5 and 2:1, between 4:5 and 2:1, between 1:1 and 2:1, between 1:10 and 1:1, between 1:10 and 4:5, between 1:10 and 3:5, between 1:10 and 2:5, or between 1:10 and 1:5), respectively. In some embodiments, the composition can contain the DNA-binding protein and the donor template in a molar ratio of between 10:1 and 1000:1 (e.g., between 50:1 and 1000:1, between 100:1 and 1000:1, between 200:1 and 1000:1, between 300:1 and 1000:1, between 400:1 and 1000:1, between 500:1 and 1000:1, between 600:1 and 1000:1, between 700:1 and 1000:1, between 800:1 and 1000:1, between 900:1 and 1000:1, between 10:1 and 900:1, between 10:1 and 800:1, between 10:1 and 700:1, between 10:1 and 600:1, between 10:1 and 500:1, between 10:1 and 400:1, between 10:1 and 300:1, between 10:1 and 200:1, between 10:1 and 100:1, or between 10:1 and 50:1), respectively. In some embodiments, the DNA-binding protein and the donor gRNA are in a molar ratio of between 1:10 and 2:1 (e.g., between 1:5 and 2:1, between 2:5 and 2:1, between 3:5 and 2:1, between 4:5 and 2:1, between 1:1 and 2:1, between 1:10 and 1:1, between 1:10 and 4:5, between 1:10 and 3:5, between 1:10 and 2:5, or between 1:10 and 1:5), respectively.

The RNA-guided nuclease can also be fused with a localization peptide or protein. For example, the RNA-guided nuclease can be fused with one or more nuclear localization signal (NLS) sequences, which can direct the nuclease and the RNP complexes it forms to the nucleus to modify the target nucleic acid. Examples of NLS sequences are known in the art, e.g., as described in Lange et al., J Biol Chem. 282(8):5101-5, 2007, and also include, but are not limited to, AVKRPAATKKAGQAKKKKLD (SEQ ID NO: 1), MSRRRKANPTKLSENAKKLAKEVEN (SEQ ID NO: 2), PAAKRVKLD (SEQ ID NO: 3), KLKIKRPVK (SEQ ID NO: 4), and PKKKRKV (SEQ ID NO: 5). Examples of other peptide or proteins that can be used to a RNA-guided nuclease, such as cell-penetrating peptides and cell-targeting peptides are available in the art and described, e.g., Vivés et al., Biochim Biophys Acta. 1786(2):126-38, 2008.

In some cases, a CRISPR-Cas effector protein (such as Cas9) is fused to one or more nuclear localization signals (NLSs) (e.g., in some cases 2 or more, 3 or more, 4 or more, or 5 or more NLSs, e.g., 1 NLS, 2 NLSs, 3 NLSs, 4 NLSs, 5 NLSs, 6 NLSs, or 7 NLSs). In some cases, one or more NLSs (e.g., 2 or more, 3 or more, 4 or more, or 5 or more NLSs) are positioned at or near (e.g., within 50 amino acids of) the N-terminus and/or the C-terminus. In some cases, one or more NLSs (2 or more, 3 or more, 4 or more, or 5 or more NLSs) are positioned at or near (e.g., within 50 amino acids of) the N-terminus. In some cases, one or more NLSs (2 or more, 3 or more, 4 or more, or 5 or more NLSs) are positioned at or near (e.g., within 50 amino acids of) the C-terminus. In some cases, one or more NLSs (3 or more, 4 or more, or 5 or more NLSs) are positioned at or near (e.g., within 50 amino acids of) both the N-terminus and the C-terminus. In some cases, an NLS is positioned at the N-terminus and an NLS is positioned at the C-terminus. In some cases, a CRISPR-Cas effector protein (such as Cas9) is fused to 1-5 NLSs (e.g., 1-4, 1-3, 1-2, 2-5, 2-5, 2-3, 3-5, or 3-4 NLSs).

Non-limiting examples of NLSs include an NLS sequence derived from: the NLS of the SV40 virus large T-antigen, having the amino acid sequence PKKKRKV (SEQ ID NO:5); the NLS from nucleoplasmin (e.g., the nucleoplasmin bipartite NLS with the sequence KRPAATKKAGQAKKKK (SEQ ID NO:6)); the c-myc NLS having the amino acid sequence PAAKRVKLD (SEQ ID NO:3) or RQRRNELKRSP (SEQ ID NO:7); the hRNPA1 M9 NLS having the sequence NQSSNFGPMKGGNFGGRSSGPYGGGGQYFAKPRNQGGY (SEQ ID NO:8); the sequence RMRIZFKNKGKDTAELRRRRVEVSVELRKAKKDEQILKRRNV (SEQ ID NO:9) of the IBB domain from importin-alpha; the sequences VSRKRPRP (SEQ ID NO:10) and PPKKARED (SEQ ID NO:11) of the myoma T protein; the sequence PQPKKKPL (SEQ ID NO:12) of human p53; the sequence SALIKKKKKMAP (SEQ ID NO:13) of mouse c-abl IV; the sequences DRLRR (SEQ ID NO:14) and PKQKKRK (SEQ ID NO:15) of the influenza virus NS1; the sequence RKLKKKIKKL (SEQ ID NO:16) of the Hepatitis virus delta antigen; the sequence REKKKFLKRR (SEQ ID NO:17) of the mouse Mx1 protein; the sequence KRKGDEVDGVDEVAKKKSKK (SEQ ID NO:18) of the human poly(ADP-ribose) polymerase; and the sequence RKCLQAGMNLEARKTKK (SEQ ID NO:19) of the steroid hormone receptors (human) glucocorticoid.

### Single-Guide RNAs

A Cas protein is guided to its target DNA by a guide RNA. In some cases, the guide RNA is a single-guide RNA (sgRNA). A sgRNA is a version of the naturally occurring two-piece guide RNA (also referred to as a dual-guide RNA) (crRNA and tracrRNA) engineered into a single, continuous sequence. A guide RNA (and also a sgRNA) includes a guide sequence (also referred to as a targeting sequence or a spacer) (e.g., equivalent to a portion of the crRNA) that targets the Cas protein to the target DNA and a scaffold sequence (also referred to as a handle or protein-binding segment or repeat) that interacts with the Cas protein (e.g., the region where a crRNA and tracrRNA hybridize to one another). A guide RNA (e.g., sgRNA) may be selected using software. As a non-limiting example, considerations for selecting a guide RNA (e.g., a sgRNA) can include, e.g., the PAM sequence for the CRISPR-Cas effector protein (e.g., Cas9 protein, Cas12a protein, etc.) to be used, and strategies for minimizing off-target modifications. Tools, such as NUPACK® and the CRISPR Design Tool, can provide sequences for preparing the sgRNA, for assessing target modification efficiency, and/or assessing cleavage at off-target sites.

### Guide Sequence

The guide sequence in the guide RNA (e.g., sgRNA) may be complementary to a specific sequence (a target sequence) within a target DNA. As would be known to one of ordinary skill in the art, a PAM sequence is located adjacent to the target sequence, in some cases 3′ of the target sequence (e.g., when a Cas9 is used) and in some cases 5′ of the target sequence, depending on which type of CRISPR-Cas effector protein is used. In general, a Cas9 protein or a variant thereof cleaves about three nucleotides upstream of the PAM sequence. The guide sequence in the guide RNA (e.g., sgRNA) can be complementary to either strand of the target DNA.

In some embodiments, the guide sequence of an guide RNA (e.g., sgRNA) includes 17-30 nucleotides (nt) (e.g., 17-25, 17-23, 17-22, 17-21, 17-20, 17-19, 17-18, 18-30, 18-25, 18-23, 18-22, 18-21, 18-20, 18-19, 19-30, 19-25, 19-23, 19-22, 19-21, 19-20, 20-30, 20-25, 20-23, 20-22, or 20-21) that can direct the Cas protein to the target DNA site using RNA-DNA complementarity base pairing. In some embodiments, the guide sequence of a guide RNA (e.g., sgRNA) is about 20 nt long. In some embodiments, the guide sequence of a guide RNA (e.g., sgRNA) is about 21 nt long. In some embodiments, the guide sequence of a guide RNA (e.g., sgRNA) is about 22 nt long. In some embodiments, the guide sequence of a guide RNA (e.g., sgRNA) is about 17 nt long. In some embodiments, the guide sequence of a guide RNA (e.g., sgRNA) is about 18 nt long. In some embodiments, the guide sequence of a guide RNA (e.g., sgRNA) is about 19 nt long. In some embodiments, the guide sequence is less than 20 nt, e.g., 19, 18, 17, 16, 15 nt or less, that are complementary to the target DNA site. In some instances, the guide sequence in the sgRNA contains at least one nucleic acid mismatch in the complementarity region of the target DNA site. In some instances, the guide sequence contains about 1 to about 10 nucleic acid mismatches in the complementarity region of the target DNA site.

### Scaffold Sequence

The scaffold sequence in the sgRNA may serve as a protein-binding sequence that interacts with the Cas protein or a variant thereof. In some embodiments, the scaffold sequence in the sgRNA can comprise two complementary stretches of nucleotides that hybridize to one another to form a double-stranded RNA duplex (dsRNA duplex). The scaffold sequence may have structures such as lower stem, bulge, upper stem, nexus, and/or hairpin. In some embodiments, the scaffold sequence in the sgRNA can be between about 90 nucleic acids to about 120 nucleic acids, e.g., about 90 nucleic acids to about 115 nucleic acids, about 90 nucleic acids to about 110 nucleic acids, about 90 nucleic acids to about 105 nucleic acids, about 90 nucleic acids to about 100 nucleic acids, about 90 nucleic acids to about 95 nucleic acids, about 95 nucleic acids to about 120 nucleic acids, about 100 nucleic acids to about 120 nucleic acids, about 105 nucleic acids to about 120 nucleic acids, about 110 nucleic acids to about 120 nucleic acids, or about 115 nucleic acids to about 120 nucleic acids.

Target gRNA and Donor gRNA

Guide RNAs (gRNAs) in general refer to a DNA-targeting RNA (a guide RNA) containing: (1) a guide sequence that is complementary to a target nucleic acid and guides the RNA-guided nuclease to the target nucleic acid and (2) a scaffold sequence that interacts and binds with the RNA-guided nuclease. In some embodiments of the disclosure, the target gRNA and the donor gRNA have the same sequence (i.e., a given guide RNA serves as both the target gRNA and the donor gRNA, e.g., this can be done when a tCTS is used as part of the donor). In other embodiments of the disclosure, the target gRNA and the donor gRNA have different sequences. In the compositions and methods described herein, a target gRNA comprises a portion (a guide sequence) that is complementary to the target nucleic acid. Once the target gRNA forms an RNP complex with the targetable nuclease (e.g., a first RNA-guided nuclease such as a Cas9), the RNP complex can be guided to the target nucleic acid by the complementarity between the target gRNA and the target nucleic acid. In some embodiments, the targetable nuclease is a Cas9 protein. The Cas9 protein identifies the target nucleic acid by first identifying a protospacer adjacent motif (PAM) located 3′ of the target nucleic acid. Once the PAM is identified, the target gRNA in the RNP complex hybridizes to the target nucleic acid upstream of the PAM. In some embodiments, a target gRNA includes a portion of nucleotides that are complementary to a portion in the target nucleic acid that is approximately 20 nucleotides upstream of the PAM sequence. In general, a Cas9 protein or a variant thereof (e.g., a nickase) cleaves about three nucleotides upstream of the PAM sequence. A gRNA can be selected using a software. As a non-limiting example, considerations for selecting a gRNA can include, e.g., the PAM sequence for the RNA-guided nuclease to be used, and strategies for minimizing off-target modifications. Tools, such as NUPACK® and the CRISPR Design Tool, can provide sequences for preparing the gRNA, for assessing target modification efficiency, and/or assessing cleavage at off-target sites.

In some embodiments, the target gRNA comprises a portion (a guide sequence) of at least 15 nucleotides (e.g., 15, 16, 17, 18, 19, 20, 21, 22, 23, 24, or 25 nucleotides) that are complementary to the target nucleic acid. In some embodiments, the target gRNA can be completely complementary or partially complementary to the target nuclei acid. In some embodiments, at least 60% (e.g., 65%, 70%, 75%, 80%, 85%, 90%, 95%, or 97%) of the nucleotides in the target nucleic acid can engage in Watson-Crick base pairing with their corresponding nucleotides in the target gRNA.

Similar to the target gRNA, the donor gRNA forms a complex with the DNA-binding protein (e.g., a RNA-guided nuclease). In the compositions and methods described herein, a donor gRNA comprises a portion (a guide sequence) that is complementary to the DNA-binding protein target sequence, which is at one or both termini of the HDR template in the donor template. Once the donor gRNA forms an RNP complex with the DNA-binding protein (e.g., a RNA-guided nuclease such as a nuclease dead Cas9, i.e., dCas9), the RNP complex can be guided to the donor template by the complementarity between the donor gRNA and the DNA-binding protein target sequence. In some embodiments, the DNA-binding protein target sequence is complementary to an equal length portion of the guide sequence of the donor gRNA (e.g., when a nuclease dead DNA-binding protein is used such as a dCas9). In some embodiments, the DNA-binding protein target sequence is complementary to a truncated portion of the guide sequence of the donor gRNA (e.g., when an active CRISPR-Cas protein such as an active Cas9 is used). In some embodiments, the donor gRNA has a guide sequence of at least 15 nucleotides (e.g., 15, 16, 17, 18, 19, 20, 21, 22, 23, 24, or 25 nucleotides), but only a portion of the guide sequence can hybridize to the DNA-binding protein target sequence. The donor gRNA can be completely complementary or partially complementary to the DNA-binding protein target sequence. In some embodiments, at least 60% (e.g., 65%, 70%, 75%, 80%, 85%, 90%, 95%, or 97%) of the nucleotides in the DNA-binding protein target sequence can engage in Watson-Crick base pairing with their corresponding nucleotides in the donor gRNA. In some embodiments, the DNA-binding protein target sequence can have at least one (e.g., one, two, three, four, five, six, seven, eight, nine, or ten) mismatched nucleotide to its corresponding nucleotide in the donor gRNA when the DNA-binding protein target sequence and the donor gRNA are hybridized. Examples of mismatched bases include a guanine and uracil, guanine and thymine, and adenine and cytosine pairing.

As described in detail further herein, the DNA-binding protein target sequence and the PAM on one or both of the region 5′ and the region 3′ (e.g., the 5′ and 3′ termini) of the HDR template can be designed with different configurations. The complex containing the DNA-binding protein (e.g., a RNA-guided nuclease), the donor gRNA, and the donor template can shuttle the donor template, without cleavage of the DNA-binding protein target sequence, to the desired intracellular location (e.g., the nucleus) such that the HDR template can integrate into the cleaved target nucleic acid.

In some embodiments of the disclosure, the target gRNA and the donor gRNA have the same sequence (i.e., the same guide RNA serves as both) and each of the targetable nuclease and the DNA-binding protein is an RNA-guided nuclease (e.g., a Cas protein such as Cas9). In this case, the gRNA can form a first RNP complex with the RNA-guided nuclease. The first RNP complex can bind to the target nucleic acid via the hybridization between the gRNA and the target nucleic acid. The gRNA can also form a second RNP complex with the RNA-guided nuclease (e.g., one that is nuclease dead, e.g., a dCas9) and the donor template. In this second RNP complex, the gRNA can bind to the DNA-binding protein target sequence in the donor template to bring the donor template to the desired intracellular location (e.g., the nucleus) for homologous recombination to occur at the cleaved target nucleic acid. In some embodiments, the guide sequence of the gRNA and the DNA-binding protein target sequence only have partial complementarity. In some embodiments, the DNA-binding protein target sequence hybridizes to a portion of the guide sequence of the donor gRNA, e.g., the DNA-binding protein target sequence is truncated or includes mismatches compared to the guide sequence of the donor gRNA. In some cases, the DNA-binding protein target sequence has the same sequence as a portion of the target nucleic acid. In some cases, the DNA-binding protein target sequence is a truncated CRISPR-Cas targeting (e.g., truncated CRISPR-Cas9 targeting) sequence (tCTS), wherein the DNA-binding protein target sequence has the same sequence as a portion of the target sequence of the target nucleic acid and thereby hybridizes to a portion of the guide sequence of the donor gRNA.

In some cases, one active CRISPR-Cas effector protein (e.g., a fully active Cas9 or a nickase Cas9) is used and serves as both the targetable nuclease and the DNA-binding protein. In such cases, a guide RNA that can serve as both the target gRNA and the donor gRNA can be used, and a tCTS can be used as part of the donor such that the active CRISPR-Cas effector protein (e.g., a fully active Cas9 or a nickase Cas9) can be targeted to the target sequence of the target DNA for cleavage, can be targeted to the donor template but does not cleave the donor (instead, the RNP bound to the donor template can serve to help the donor template localize to a location in the cell, e.g., the nucleus, by way of the Cas9 protein including one or more NLSs).

### Donor Template

The HDR template, one or more (e.g., one, two, three, four, five, six, seven, eight, nine, or ten) DNA-binding protein target sequences, and one or more (e.g., one, two, three, four, five, six, seven, eight, nine, or ten) PAMs (when present) can have several different configurations in the donor template to enhance homology directed repair between the HDR template and the target nucleic acid. The donor template can also contain one or more edge sequences to facilitate the binding of the DNA-binding protein. In one example, the donor template contains one DNA-binding protein target sequence and one PAM. In some embodiments of this example, the DNA-binding protein target sequence and the PAM are located in the region 5′ of the HDR template (e.g., in some cases at or near the 5′ terminus of the HDR template, i.e., within 100 nucleotides, e.g., 50, 40, or 30 nucleotides of the 5′ terminus). In particular, when both the DNA-binding protein target sequence and the PAM are located in the region 5′ of the HDR template, the PAM can be located adjacent and 5′ or 3′ of the DNA-binding protein target sequence (e.g., depending on the type of CRISPR-Cas protein that is used). In other embodiments, the DNA-binding protein target sequence and the PAM are located in the region 3′ of the HDR template (e.g., in some cases at or near the 3′ terminus of the HDR template, i.e., within 100 nucleotides, e.g., 50, 40, or 30 nucleotides of the 3′ terminus). In particular, when both the DNA-binding protein target sequence and the PAM are located in the region 3′ of the HDR template, the PAM can be located adjacent and 5′ or 3′ of the DNA-binding protein target sequence (e.g., depending on the type of CRISPR-Cas protein that is used). In some embodiments, a DNA-binding protein target sequence and a PAM are located in both in the region 5′ of the HDR template (e.g., in some cases at or near the 5′ terminus of the HDR template, i.e., within 100 nucleotides, e.g., 50, 40, or 30 nucleotides of the 5′ terminus) and in the region 3′ of the HDR template (e.g., in some cases at or near the 3′ terminus of the HDR template, i.e., within 100 nucleotides, e.g., 50, 40, or 30 nucleotides of the 3′ terminus). In particular, when both a DNA-binding protein target sequence and a PAM are located in both the region 5′ of the HDR template and in the region 3′ of the HDR template, each PAM can be located adjacent to and 5′ or 3′ of the DNA-binding protein target sequence (e.g., depending on the type of CRISPR-Cas protein that is used).

In some embodiments, the length (also referred to as the size) of the donor template is greater than about 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 1 kb, 1.1 kb, 1.2 kb, 1.3 kb, 1.4 kb, 1.5 kb, 1.6 kb, 1.7 kb, 1.8 kb, 1.9 kb, 2.0 kb, 2.1 kb, 2.2 kb, 2.3 kb, 2.4 kb, 2.5 kb, 2.6 kb, 2.7 kb, 2.8 kb, 2.9 kb, 3 kb, 3.1 kb, 3.2 kb, 3.3 kb, 3.4 kb, 3.5 kb, 3.6 kb, 3.7 kb, 3.8 kb, 3.9 kb, 4.0 kb, 4.1 kb, 4.2 kb, 4.3 kb, 4.4 kb, 4.5 kb, 4.6 kb, 4.7 kb, 4.8 kb, 4.9 kb, 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.0 kb, 8.1 kb, 8.2 kb, 8.3 kb, 8.4 kb, 8.5 kb, 8.6 kb, 8.7 kb, 8.8 kb, 8.9 kb, 9.0 kb, 9.1 kb, 9.2 kb, 9.3 kb, 9.4 kb, 9.5 kb, 9.6 kb, 9.7 kb, 9.8 kb, 9.9 kb, 10.0 kb, any size of template in between these sizes, or greater than 10 kb. For example, the size of the template can be about 200 bp to about 500 bp, about 200 bp to about 750 bp, about 200 bp to about 1 kb, about 200 bp to about 1.5 kb, about 200 bp to about 2.0 kb, about 200 bp to about 2.5 kb, about 200 bp to about 3.0 kb, about 200 bp to about 3.5 kb, about 200 bp to about 4.0 kb, about 200 bp to about 4.5 kb, about 200 bp to about 5.0 kb. In some cases, the size of the template is large enough and in sufficient quantity to be lethal as naked DNA.

In some embodiments, the donor template encodes a heterologous protein or a fragment thereof. In some embodiments, the template includes regulatory sequences, for example, a promoter sequence and/or an enhancer sequence to regulate expression of the heterologous protein or fragment thereof, e.g., after insertion into the genome of a cell. A heterologous protein can include a chimeric antigen receptor (CAR). As such, a CAR-T cell can be generated. A heterologous protein can include a T cell receptor (TCR). As such, a TCR-T cell can be generated.

In some embodiments, the donor template includes an exogenous sequence such as an exogenous nucleotide sequence. An exogenous sequence can include an encoded heterologous protein or a fragment thereof. An exogenous sequence can include a gene or portion thereof. An exogenous nucleotide sequence can be a short sequence, e.g., of 3-100 nucleotides in length. An exogenous nucleotide sequence of interest can be a single nucleotide. In addition, an exogenous nucleotide sequence of interest can be a long sequence, e.g., of 500-3000 nucleotides in length. An exogenous nucleotide sequence of interest can be coding or non-coding for a polypeptide sequence. In addition, an exogenous nucleotide sequence of interest can be inserted in a cell such that it forms a chimeric gene upon insertion. For example, an exogenous receptor portion can be inserted in frame in an endogenous receptor coding sequence to produce a chimeric receptor coding sequence that, post-editing, includes the exogenous receptor portion operably linked to an endogenous intracellular portion (e.g., for signal transduction).

In some examples, a gene or portion thereof can be a protein-coding nucleotide sequence (i.e., a nucleotide sequence encoding a polypeptide sequence). In general, any protein coding nucleotide can be used. In some examples, a protein coding nucleotide sequence encodes a protein useful in autologous cell therapies (e.g., autologous T cell therapies). In some examples, a protein coding nucleotide sequence can include, but is not limited to, a factor that modulates the immune system, a cytokine, a factor that modulates T cell function, a factor that promotes T-cell survival, a factor that promotes T-cell function, or an immune checkpoint inhibitor. A protein coding nucleotide sequence, particularly a secreted protein or membrane-bound proteins, can include a nucleotide sequence encoding a signal peptide. The signal peptide can be endogenous to the protein encoded by the protein coding nucleotide sequence. The signal peptide can be exogenous to the protein encoded by the protein coding nucleotide sequence.

In some examples, a gene or portion thereof can be a non-protein coding nucleotide sequence. In general, any non-protein coding nucleotide can be used. In some cases, a non-protein coding nucleotide sequence can be a nucleotide sequence useful in autologous cell therapies (e.g., autologous T cell therapies). In some cases, a non-protein coding nucleotide sequence can include, but is not limited to, an shRNA, an siRNA, an miRNA, and an lncRNA.

Although a nucleotide sequence encoding at least a portion of a gene (e.g., an exogenous gene of interest) can, in general, be any size, practical considerations, such as the impact of gene size on overall template size and on subsequent overall editing efficiency, can be taken into account. Thus, in a particular aspect, provided herein are modified cells that are genomically edited, or are capable of being genomically edited, to express an exogenous gene greater than or equal to 100 bases in length at HR efficiency rates greater than those previously described (e.g., a greater percentage of a population having an integrated polynucleotide sequence), particularly when using non-viral delivery methods. The improved HR efficiency rates similarly apply to genes greater than 100 bases in length, such as introducing exogenous sequences greater than or equal to 200 bases in length, greater than or equal to 400 bases in length, greater than or equal to 500 bases in length, greater than or equal to 600 bases in length, greater than or equal to 750 bases in length, greater than or equal to 1000 bases in length greater than or equal to 1500 bases in length, greater than or equal to 2000 bases in length, greater than or equal to 3000 bases in length, or greater than or equal to 4000 bases in length. The at least a portion of a gene can be greater than or equal to 800 bases in length. The at least a portion of a gene can be greater than or equal to 1600 bases in length.

Exogenous sequences can be between 100-200 bases in length, between 100-300 bases in length, between 100-400 bases in length, between 100-500 bases in length, between 100-600 bases in length, between 100-700 bases in length, between 100-800 bases in length, between 100-900 bases in length, or between 100-1000 bases in length. Exogenous sequences can be between 100-2000 bases in length, between 100-3000 bases in length, between 100-4000 bases in length, between 100-5000 bases in length, between 100-6000 bases in length, between 100-7000 bases in length, between 100-8000 bases in length, between 100-9000 bases in length, or between 100-10,000 bases in length. Exogenous sequences can be between 1000-2000 bases in length, between 1000-3000 bases in length, between 1000-4000 bases in length, between 1000-5000 bases in length, between 1000-6000 bases in length, between 1000-7000 bases in length, between 1000-8000 bases in length, between 1000-9000 bases in length, or between 1000-10,000 bases in length.

Exogenous sequences can be greater than or equal to 10 bases in length, greater than or equal to 20 bases in length, greater than or equal to 30 bases in length, greater than or equal to 40 bases in length, greater than or equal to 50 bases in length, greater than or equal to 60 bases in length, greater than or equal to 70 bases in length, greater than or equal to 80 bases in length greater than or equal to 90 bases in length, or greater than or equal to 95 bases in length. Exogenous sequences can be between 1-100 bases in length, between 1-90 bases in length, between 1-80 bases in length, between 1-70 bases in length, between 1-60 bases in length, between 1-50 bases in length, between 1-40 bases in length, or between 1-30 bases in length. Exogenous sequences can be between 1-20 bases in length, between 2-20 bases in length, between 3-20 bases in length, between 5-20 bases in length, between 10-20 bases in length, or between 15-20 bases in length. Exogenous sequences can be between 1-10 bases in length, between 2-10 bases in length, between 3-10 bases in length, between 5-10 bases in length, between 1-5 bases in length, or between 1-15 bases in length. Exogenous sequences can be 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 115, 120, 125, 150, 175, 200, 225, or 250 bases in length. Exogenous sequences can be 1, 2, 3, 4, 5, 6, 7, 8, 9, 10, 11, 12, 13, or 14 bases in length. Exogenous sequences can be greater than about 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 1 kb, 1.1 kb, 1.2 kb, 1.3 kb, 1.4 kb, 1.5 kb, 1.6 kb, 1.7 kb, 1.8 kb, 1.9 kb, 2.0 kb, 2.1 kb, 2.2 kb, 2.3 kb, 2.4 kb, 2.5 kb, 2.6 kb, 2.7 kb, 2.8 kb, 2.9 kb, 3 kb, 3.1 kb, 3.2 kb, 3.3 kb, 3.4 kb, 3.5 kb, 3.6 kb, 3.7 kb, 3.8 kb, 3.9 kb, 4.0 kb, 4.1 kb, 4.2 kb, 4.3 kb, 4.4 kb, 4.5 kb, 4.6 kb, 4.7 kb, 4.8 kb, 4.9 kb, 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb or any size of template in between these sizes.

In examples where multiple exogenous sequences are introduced, the multiple exogenous sequences can be different sizes, e.g., a first exogenous sequence can be greater than or equal to 100 bases and a second exogenous sequence can be greater than or equal to 100 bases, or a first exogenous sequence can be greater than or equal to 100 bases and a second exogenous sequence can be less than 100 bases (e.g., between 1-100 bases in length).

In some cases, the donor template is a linear DNA template. In some cases, the donor template is a double-stranded DNA (dsDNA). In some cases, the donor template is a single-stranded DNA (ssDNA). In some cases, the donor template includes both ssDNA and dsDNA (which can be referred to as a ssDNA/dsDNA hybrid donor template). For example, in some cases, one or more oligonucleotides can be added that hybridize to the one or more DNA-binding protein target sequences to create a double stranded region for the DNA-binding protein to bind. For example, in some cases, the donor template is an ssDNA, but oligonucleotides can be included (rendering the donor template double stranded in regions where the oligonucleotides hybridize) to provide double stranded regions for a CRISPR-Cas effector protein (e.g., a Cas9 protein) to bind, e.g., the double stranded regions can include PAM sites.

In some cases, the single-stranded DNA template is a pure single-stranded DNA template. As used herein, by “pure single-stranded DNA” is meant single-stranded DNA that substantially lacks the other or opposite strand of DNA. By “substantially lacks” is meant that the pure single-stranded DNA lacks at least 100-fold more of one strand than another strand of DNA. In some cases, the donor template is a double-stranded plasmid. In some cases the donor template is a single-stranded plasmid. In some cases the donor template is a mini-circle.

A donor template can be non-viral. A template can be a plasmid. A template can be a minicircle. A template can be a nanoplasmid. A template can be circular.

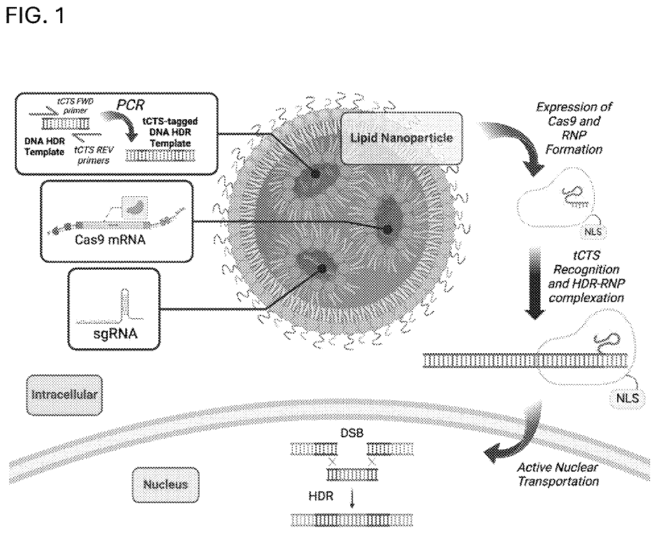

The one or more DNA-binding protein target sequence(s), and PAM sequence(s) if present, can be introduced into dsDNA templates of any format, including linear dsDNA sequences produced by PCR, restriction enzyme digestions, or any other linearization method, as well as circular dsDNA sequences such as plasmids. In the case of a plasmid, the one or more DNA-binding protein target sequence(s) can be cloned into the plasmid outside of the homology arms and DNA insert regions, including but not limited to adjacent to the edge(s) of the homology arm(s). Similar to linear dsDNA templates, the DNA binding protein complex (e.g., RNP made from Cas9 and gRNA) can be incubated briefly with plasmid DNA template to allow for binding of the DNA plasmid by the RNP prior to introduction into the cell (e.g., via electroporation). See, e.g., FIGS. 1, 2, and 10B of International Patent Publication No. WO2018232356 and paragraph [0100] of International Patent Publication No. WO2019084552. Plasmid templates with shuttle sequence(s) can also be used with one or more lipid nanoparticles, e.g., as described herein.

In some embodiments, the donor template contains two DNA-binding protein target sequences and two PAMs. In some embodiments of this example, a first DNA-binding protein target sequence and a first PAM are located in a region 5′ of the HDR template (e.g., at the 5′ terminus of the HDR template) and a second DNA-binding protein target sequence and a second PAM are located in a region 3′ of the HDR template (e.g., at the 3′ terminus of the HDR template). Further, in some embodiments, a first PAM can be located adjacent the first DNA-binding protein target sequence and a second PAM (in some cases the same sequence as the first PAM) can be located adjacent the second DNA-binding protein target sequence. In some embodiments, the first PAM can be located adjacent and 5′ of the first DNA-binding protein target sequence and the second PAM can be located adjacent and 5′ of the second DNA-binding protein target sequence. In some embodiments, the first PAM can be located adjacent and 3′ of the first DNA-binding protein target sequence and the second PAM can be located adjacent and 3′ of the second DNA-binding protein target sequence. In some embodiments, the first PAM can be located adjacent and 5′ of the first DNA-binding protein target sequence and the second PAM can be located adjacent and 3′ of the second DNA-binding protein target sequence.

In some embodiments, one or more DNA-binding protein target sequences and one or more PAMs can be located within the HDR template as long as they do not interfere with the homology directed repair between the HDR template and the target nucleic acid.

The donor template can further contain one or more edge sequences at either or both of the 5′ and 3′ termini of the donor template. An edge sequence in the donor template can facilitate binding between the donor template and the DNA-binding protein (e.g., an RNA-guided nuclease). In some embodiments, an edge sequence can have at least 2 nucleotides, e.g., between 2 and 24 nucleotides (e.g., between 2 and 22, between 2 and 20, between 2 and 18, between 2 and 16, between 2 and 14, between 2 and 12, between 2 and 10, between 2 and 8, between 2 and 6, between 2 and 4, between 4 and 24, between 6 and 24, between 8 and 24, between 10 and 24, between 12 and 24, between 14 and 24, between 16 and 24, between 18 and 24, between 20 and 24, or between 22 and 24 nucleotides; 2, 3, 4, 5, 6, 7, 8, 9, 10, 11, 12, 13, 14, 15, 16, 17, 18, 19, 20, 21, 22, 23, or 24 nucleotides).

### Targetable Nuclease

As described above, in some embodiments of the compositions and methods described herein, the targetable nuclease is an RNA-guided nuclease (e.g., a CRISPR-Cas effector protein). In some cases, the targetable nuclease is provided as an mRNA encoding the targetable nuclease. In some embodiments, the targetable nuclease can recognize a sequence of a target nucleic acid (e.g., a target gene within a genome), bind to the target nucleic acid, and modify the target nucleic acid. In other embodiments, the targetable nuclease can be a fusion protein that includes a protein that can bind to the target nucleic acid and a protein that can modify the target nucleic acid (e.g., a nuclease, a transcription activator or repressor).

In some embodiments, the targetable nuclease has nuclease activity, i.e., can modify the target nucleic acid by cleaving the target nucleic acid (in some cases double stranded cleavage and in some cases single stranded cleavage, i.e., nicking). In some cases, the cleaved target nucleic acid can then undergo homologous recombination with a nearby a homology directed repair (HDR) template. For example, the Cas nuclease can direct cleavage of one or both strands at a location in a target nucleic acid.

In some cases, the CRISPR-Cas effector polypeptide is a type II CRISPR-Cas effector polypeptide. In some cases, the type II CRISPR-Cas effector polypeptide is a Cas9 polypeptide. In some cases, the CRISPR-Cas effector polypeptide is a type V CRISPR-Cas effector polypeptide, e.g., a Cas12a, a Cas12b, a Cas12c, a Cas12d, or a Cas12e polypeptide. In some cases, the CRISPR-Cas effector polypeptide is a type VI CRISPR-Cas effector polypeptide, e.g., a Cas13a polypeptide, a Cas13b polypeptide, a Cas13c polypeptide, or a Cas13d polypeptide. In some cases, the CRISPR-Cas effector polypeptide is a Cas14 polypeptide. In some cases, the CRISPR-Cas effector polypeptide is a Cas14a polypeptide, a Cas14b polypeptide, or a Cas14c polypeptide. Also suitable for use is a variant CRISPR-Cas effector polypeptide, where the variant CRISPR-Cas effector polypeptide has reduced nucleic acid cleavage activity. Examples of CRISPR-Cas effector polypeptides are CRISPR-Cas endonucleases (e.g., class 2 CRISPR-Cas effector polypeptide such as a type II, type V, or type VI CRISPR-Cas effector polypeptide). Where a CRISPR-Cas effector polypeptide has endonuclease activity, the CRISPR-Cas effector polypeptide may also be referred to as a “CRISPR-Cas endonuclease.” A CRISPR-Cas effector polypeptide can also have nickase activity. CRISPR-Cas effector proteins are known to those skilled in the art. For example, an amino acid sequence of a Streptococcus pyogenes wild-type Cas9 polypeptide is set forth, e.g., in NBCI Ref. Seq. No. WP_219102679.1, and the amino acid sequence of a Streptococcus thermophilus wild-type Cas9 polypeptide is set forth, e.g., in NBCI Ref. Seq. No. WP_011681470.

Cas nucleases, e.g., Cas9 nucleases, can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. multocida, Sutterella wadsworthensis, proteobacterium, Legionella pneumophila, Parasutterella excrementihominis, Wolinella succinogenes, and Francisella novicida.

Cas9 protein refers to an RNA-guided double-stranded DNA-binding nuclease protein or nickase protein. Wild-type Cas9 nuclease has two functional domains, e.g., RuvC and HNH, that cut different DNA strands. Cas9 can induce double-strand breaks in genomic DNA (target DNA) when both functional domains are active. The Cas9 enzyme can comprise one or more catalytic domains of a Cas9 protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the Cas9 can be a fusion protein, e.g., the two catalytic domains are derived from different bacteria species.

In some embodiments, a Cas protein can be a Cas protein variant. For example, useful variants of the Cas9 nuclease can include a single inactive catalytic domain, such as a RuvC- or HNH-enzyme or a nickase. A Cas9 nickase has only one active functional domain and can cut only one strand of the target DNA, thereby creating a single strand break or nick. In some embodiments, the Cas9 nuclease can be a mutant Cas9 nuclease having one or more amino acid mutations. For example, the mutant Cas9 having at least a D10A mutation is a Cas9 nickase. In other embodiments, the mutant Cas9 nuclease having at least a H840A mutation is a Cas9 nickase. Other examples of mutations present in a Cas9 nickase include, without limitation, N854A and N863A. A double-strand break can be introduced using a Cas9 nickase if at least two DNA-targeting RNAs that target opposite DNA strands are used. A double-nicked induced double-strand break can be repaired by NHEJ or HDR (Ran et al., 2013, Cell, 154:1380-1389). Non-limiting examples of Cas9 nucleases or nickases are described in, for example, U.S. Pat. Nos. 8,895,308; 8,889,418; and 8,865,406 and U.S. Application Publication Nos. 2014/0356959, 2014/0273226 and 2014/0186919. The Cas9 nuclease or nickase can be codon-optimized for the target cell or target organism.

In some embodiments, a Cas protein variant that lacks cleavage (e.g., nickase) activity. A Cas protein variant may contain one or more point mutations that eliminates the protein's nickase activity. In some embodiments, such Cas protein variants can be fused to other proteins and serve as targeting domains to direct the other proteins to the target nucleic acid. For example, Cas protein variants without nickase activity may be fused to transcriptional activation or repression domains to control gene expression (Ma et al., Protein and Cell, 2(11):879-888, 2011; Maeder et al., Nature Methods, 10:977-979, 2013; and Konermann et al., Nature, 517:583-588, 2014). A Cas protein variant that lacks nickase activity may be used to target genomic regions, resulting in RNA-directed transcriptional control. In some embodiments, a Cas protein variant without any cleavage (e.g., nickase) activity may be used to target an exogenous protein to the target nucleic acid. An exogenous protein may be fused to the Cas protein variant and the fusion protein may be enhanced by the addition of the the lipid nanoparticle. An exogenous protein may be an effector protein domain. An exogenous protein may be a transcription activator or repressor. Other examples of exogenous proteins include, but are not limited to, VP64-p65-Rta (VPR), VP64, P65, Krab, Ten-eleven translocation methylcytosine dioxygenase (TET), and DNA methyltransferase (DNMT). Specific Cas protein variants that lack cleavage (e.g., nickase) activity are also described below.

In some embodiments, the Cas nuclease can be a high-fidelity or enhanced specificity Cas9 polypeptide variant with reduced off-target effects and robust on-target cleavage. Non-limiting examples of Cas9 polypeptide variants with improved on-target specificity include the SpCas9 (K855A), SpCas9 (K810A/K1003A/R1060A) (also referred to as eSpCas9(1.0)), and SpCas9 (K848A/K1003A/R1060A) (also referred to as eSpCas9(1.1)) variants described in Slaymaker et al., Science, 351(6268):84-8 (2016), and the SpCas9 variants described in Kleinstiver et al., Nature, 529(7587):490-5 (2016) containing one, two, three, or four of the following mutations: N497A, R661A, Q695A, and Q926A (e.g., SpCas9-HF1 contains all four mutations).

In some embodiments, a targetable nuclease can also be a fusion protein that contains a protein that can bind to the target nucleic acid and a protein that can cleave the target nucleic acid. For example, a protein that can recognize and bind to the target nucleic acid can be a Cas protein variant without any cleavage activity. A Cas protein variant without any cleavage activity can be a Cas9 polypeptide that contains two silencing mutations of the RuvC1 and HNH nuclease domains (D10A and H840A), which is referred to as dCas9 (Jinek et al., Science, 2012, 337:816-821; Qi et al., Cell, 152(5):1173-1183). In one embodiment, the dCas9 polypeptide from Streptococcus pyogenes comprises at least one mutation at position D10, G12, G17, E762, H840, N854, N863, H982, H983, A984, D986, A987 or any combination thereof. Descriptions of such dCas9 polypeptides and variants thereof are provided in, for example, International Patent Publication No. WO 2013/176772. The dCas9 enzyme can contain a mutation at D10, E762, H983, or D986, as well as a mutation at H840 or N863. In some instances, the dCas9 enzyme can contain a D10A or D10N mutation. Also, the dCas9 enzyme can contain a H840A, H840Y, or H840N. In some embodiments, the dCas9 enzyme can contain D10A and H840A; D10A and H840Y; D10A and H840N; D.sub.10N and H840A; D10N and H840Y; or D10N and H840N substitutions. The substitutions can be conservative or non-conservative substitutions to render the Cas9 polypeptide catalytically inactive and able to bind to target DNA.

In other embodiments, a protein that can recognize and bind to the target nucleic acid can be a transcription activator-like (TAL) effector DNA-binding protein or a zinc finger DNA-binding protein. The TAL effector DNA-binding protein has a central domain of DNA-binding tandem repeats usually containing 33-35 amino acids in length and two hypervariable amino acid residues at positions 12 and 13 that can recognize one or more specific DNA base pairs. The zinc finger DNA-binding protein has a DNA-binding motif that is often characterized by the absence or presence one or more zinc ions in order to coordinate and stabilize the motif fold. The zinc finger DNA-binding protein contains multiple finger-like protrusions that make tandem contacts with their target molecule. Some zinc finger DNA-binding proteins also form salt bridges to stabilize the finger-like folds. They were first identified as a DNA-binding motif in transcription factor TFIIIA from Xenopus laevis (African clawed frog), however they are now recognized to bind DNA, RNA, protein, and/or lipid substrates.

In some embodiments, a targetable nuclease in the compositions and methods described herein can be a fusion protein containing a TAL effector DNA-binding protein and a protein that can cleave the target nucleic acid (also referred to as “Transcription activator-like effector nucleases (TALEN)”). In other embodiments, a targetable nuclease in the compositions and methods described herein can be a fusion protein containing a zinc finger DNA-binding protein and a protein that can cleave the target nucleic acid. For example, a protein that can cleave the target nucleic acid can be a wild-type or mutated FokI endonuclease or the catalytic domain of FokI. Detailed descriptions of TALENs and their uses for gene editing are found, e.g., in U.S. Pat. Nos. 8,440,431; 8,440,432; 8,450,471; 8,586,363; and U.S. Pat. No. 8,697,853; Scharenberg et al., Curr Gene Ther, 2013, 13(4):291-303; Gaj et al., Nat Methods, 2012, 9(8):805-7; Beurdeley et al., Nat Commun, 2013, 4:1762; and Joung and Sander, Nat Rev Mol Cell Biol, 2013, 14(1):49-55. Examples of a zinc finger DNA-binding protein fused to a protein that can cleave the target nucleic acid are described in the art and include, but are not limited to, those described in Urnov et al., Nature Reviews Genetics, 2010, 11:636-646; Gaj et al., Nat Methods, 2012, 9(8):805-7; U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; and U.S. Application Publication Nos. 2003/0232410 and 2009/0203140.

In some embodiments, the targetable nuclease does not have nuclease activity. For example, the targetable nuclease (e.g., a targetable nuclease without any nuclease activity) can regulate the expression of the target nucleic acid. In some embodiments, the targetable nuclease can be a fusion protein that includes a protein that can bind to the target nucleic acid, such as a Cas protein variant without any cleavage activity (e.g., a dCas9), a TAL effector DNA-binding protein, and a zinc finger DNA-binding protein as described above, and a protein that can modify the target nucleic acid, such as a transcription activator or repressor.

The targetable nuclease can also be fused with a localization peptide or protein. For example, the targetable nuclease can be fused with one or more nuclear localization signal (NLS) sequences, which can direct the targetable nuclease and the RNP complexes it forms to the nucleus to modify the target nucleic acid. Examples of NLS sequences are known in the art, e.g., as described in Lange et al., J Biol Chem. 282(8):5101-5, 2007, and also include, but are not limited to, AVKRPAATKKAGQAKKKKLD (SEQ ID NO: 1), MSRRRKANPTKLSENAKKLAKEVEN (SEQ ID NO: 2), PAAKRVKLD (SEQ ID NO: 3), KLKIKRPVK (SEQ ID NO: 4), and PKKKRKV (SEQ ID NO: 5). Examples of other peptide or proteins that can be used to a targetable nuclease, such as cell-penetrating peptides and cell-targeting peptides are available in the art and described, e.g., Vives et al., Biochim Biophys Acta. 1786(2):126-38, 2008.

In some cases, a targetable nuclease (e.g., a CRISPR-Cas effector protein such as Cas9) is fused to one or more nuclear localization signals (NLSs) (e.g., in some cases 2 or more, 3 or more, 4 or more, or 5 or more NLSs, e.g., 1 NLS, 2 NLSs, 3 NLSs, 4 NLSs, 5 NLSs, 6 NLSs, or 7 NLSs). In some cases, one or more NLSs (e.g., 2 or more, 3 or more, 4 or more, or 5 or more NLSs) are positioned at or near (e.g., within 50 amino acids of) the N-terminus and/or the C-terminus. In some cases, one or more NLSs (2 or more, 3 or more, 4 or more, or 5 or more NLSs) are positioned at or near (e.g., within 50 amino acids of) the N-terminus. In some cases, one or more NLSs (2 or more, 3 or more, 4 or more, or 5 or more NLSs) are positioned at or near (e.g., within 50 amino acids of) the C-terminus. In some cases, one or more NLSs (3 or more, 4 or more, or 5 or more NLSs) are positioned at or near (e.g., within 50 amino acids of) both the N-terminus and the C-terminus. In some cases, an NLS is positioned at the N-terminus and an NLS is positioned at the C-terminus. In some cases, a targetable nuclease (e.g., a CRISPR-Cas effector protein such as Cas9) is fused to 1-5 NLSs (e.g., 1-4, 1-3, 1-2, 2-5, 2-5, 2-3, 3-5, or 3-4 NLSs).

Non-limiting examples of NLSs include an NLS sequence derived from: the NLS of the SV40 virus large T-antigen, having the amino acid sequence PKKKRKV (SEQ ID NO:5); the NLS from nucleoplasmin (e.g., the nucleoplasmin bipartite NLS with the sequence KRPAATKKAGQAKKKK (SEQ ID NO:6)); the c-myc NLS having the amino acid sequence PAAKRVKLD (SEQ ID NO:3) or RQRRNELKRSP (SEQ ID NO:7); the hRNPA1 M9 NLS having the sequence NQSSNFGPMKGGNFGGRSSGPYGGGGQYFAKPRNQGGY (SEQ ID NO:8); the sequence RMRIZFKNKGKDTAELRRRRVEVSVELRKAKKDEQILKRRNV (SEQ ID NO:9) of the IBB domain from importin-alpha; the sequences VSRKRPRP (SEQ ID NO:10) and PPKKARED (SEQ ID NO:11) of the myoma T protein; the sequence PQPKKKPL (SEQ ID NO:12) of human p53; the sequence SALIKKKKKMAP (SEQ ID NO:13) of mouse c-abl IV; the sequences DRLRR (SEQ ID NO:14) and PKQKKRK (SEQ ID NO:15) of the influenza virus NS1; the sequence RKLKKKIKKL (SEQ ID NO:16) of the Hepatitis virus delta antigen; the sequence REKKKFLKRR (SEQ ID NO:17) of the mouse Mx1 protein; the sequence KRKGDEVDGVDEVAKKKSKK (SEQ ID NO:18) of the human poly(ADP-ribose) polymerase; and the sequence RKCLQAGMNLEARKTKK (SEQ ID NO:19) of the steroid hormone receptors (human) glucocorticoid.

### DNA-Binding Protein

A DNA-binding protein is a protein that can directly or indirectly bind to a DNA-binding protein target sequence within a donor template (which includes an HDR template). In some cases, the DNA-binding protein is provided as an mRNA encoding the DNA-binding protein. In some embodiments, a DNA-binding protein can be an RNA-guided nuclease (e.g., a CRISPR-Cas effector protein) that can recognize and bind the DNA-binding protein target sequence, but not cleave the DNA-binding protein target sequence. The RNA-guided nuclease can bind to the DNA-binding protein target sequence via the donor gRNA as described above. In some embodiments, the donor gRNA and the DNA-binding protein target sequence can have partial complementarity which allows the RNA-guided nuclease to bind to the DNA-binding protein target sequence via the donor gRNA but not cleave the DNA-binding protein target sequence. In other embodiments, a DNA-binding protein can be a Cas protein variant without any cleavage activity (e.g., a dCas9), a TAL effector DNA-binding protein, or a zinc finger DNA-binding protein as described above. Each of the TAL effector DNA-binding protein and zinc finger DNA-binding protein can directly bind to a DNA-binding protein target sequence within a donor template. Without being bound by any theory, the DNA-binding protein serves to transport or shuttle the donor template to a cellular location close to the target nucleic acid (e.g., the nucleus).

The DNA-binding protein can also be fused with a localization peptide or protein. For example, the DNA-binding protein can be fused with one or more nuclear localization signal (NLS) sequences, which can direct the DNA-binding protein and the RNP complexes it forms to the nucleus to modify the target nucleic acid. Examples of NLS sequences are known in the art, e.g., as described in Lange et al., J Biol Chem. 282(8):5101-5, 2007, and also include, but are not limited to, AVKRPAATKKAGQAKKKKLD (SEQ ID NO: 1), MSRRRKANPTKLSENAKKLAKEVEN (SEQ ID NO: 2), PAAKRVKLD (SEQ ID NO: 3), KLKIKRPVK (SEQ ID NO: 4), and PKKKRKV (SEQ ID NO: 5). Examples of other peptide or proteins that can be used to a DNA-binding protein, such as cell-penetrating peptides and cell-targeting peptides are available in the art and described, e.g., Vives et al., Biochim Biophys Acta. 1786(2):126-38, 2008.

In some cases, a DNA-binding protein (e.g., a CRISPR-Cas effector protein such as Cas9) is fused to one or more nuclear localization signals (NLSs) (e.g., in some cases 2 or more, 3 or more, 4 or more, or 5 or more NLSs, e.g., 1 NLS, 2 NLSs, 3 NLSs, 4 NLSs, 5 NLSs, 6 NLSs, or 7 NLSs). In some cases, one or more NLSs (e.g., 2 or more, 3 or more, 4 or more, or 5 or more NLSs) are positioned at or near (e.g., within 50 amino acids of) the N-terminus and/or the C-terminus. In some cases, one or more NLSs (2 or more, 3 or more, 4 or more, or 5 or more NLSs) are positioned at or near (e.g., within 50 amino acids of) the N-terminus. In some cases, one or more NLSs (2 or more, 3 or more, 4 or more, or 5 or more NLSs) are positioned at or near (e.g., within 50 amino acids of) the C-terminus. In some cases, one or more NLSs (3 or more, 4 or more, or 5 or more NLSs) are positioned at or near (e.g., within 50 amino acids of) both the N-terminus and the C-terminus. In some cases, an NLS is positioned at the N-terminus and an NLS is positioned at the C-terminus. In some cases, a DNA-binding protein (e.g., a CRISPR-Cas effector protein such as Cas9) is fused to 1-5 NLSs (e.g., 1-4, 1-3, 1-2, 2-5, 2-5, 2-3, 3-5, or 3-4 NLSs).

Non-limiting examples of NLSs include an NLS sequence derived from: the NLS of the SV40 virus large T-antigen, having the amino acid sequence PKKKRKV (SEQ ID NO:xx); the NLS from nucleoplasmin (e.g., the nucleoplasmin bipartite NLS with the sequence KRPAATKKAGQAKKKK (SEQ ID NO:6)); the c-myc NLS having the amino acid sequence PAAKRVKLD (SEQ ID NO:3) or RQRRNELKRSP (SEQ ID NO:7); the hRNPA1 M9 NLS having the sequence NQSSNFGPMKGGNFGGRSSGPYGGGGQYFAKPRNQGGY (SEQ ID NO:8); the sequence RMRIZFKNKGKDTAELRRRRVEVSVELRKAKKDEQILKRRNV (SEQ ID NO:9) of the IBB domain from importin-alpha; the sequences VSRKRPRP (SEQ ID NO:10) and PPKKARED (SEQ ID NO:11) of the myoma T protein; the sequence PQPKKKPL (SEQ ID NO:12) of human p53; the sequence SALIKKKKKMAP (SEQ ID NO:13) of mouse c-abl IV; the sequences DRLRR (SEQ ID NO:14) and PKQKKRK (SEQ ID NO:15) of the influenza virus NS1; the sequence RKLKKKIKKL (SEQ ID NO:16) of the Hepatitis virus delta antigen; the sequence REKKKFLKRR (SEQ ID NO:17) of the mouse Mx1 protein; the sequence KRKGDEVDGVDEVAKKKSKK (SEQ ID NO:18) of the human poly(ADP-ribose) polymerase; and the sequence RKCLQAGMNLEARKTKK (SEQ ID NO:19) of the steroid hormone receptors (human) glucocorticoid.

### DNA-Binding Protein Target Sequence

A DNA-binding protein target sequence is a nucleotide sequence that is recognized and bound by a DNA-binding protein. In the compositions and methods described herein, one or more DNA-binding protein target sequences are added to an HDR template, such that the HDR template can be brought or “shuttled” into the desired intracellular location (e.g., the nucleus) to be near the target nucleic acid. Thus, the DNA-binding protein target sequence can help to improve homology directed repair efficiency and target nucleic acid modification efficiency.

In some embodiments, a DNA-binding protein target sequence can be directly recognized and bound by a DNA-binding protein, e.g., a TAL effector DNA-binding protein or zinc finger DNA-binding protein. In other embodiments, a DNA-binding protein target sequence can be indirectly recognized and bound by a DNA-binding protein, e.g., an RNA-guided nuclease, via a donor gRNA. In some embodiments, at least 60% (e.g., 65%, 70%, 75%, 80%, 85%, 90%, 95%, or 97%) of the nucleotides in the DNA-binding protein target sequence can engage in Watson-Crick base pairing with their corresponding nucleotides in the donor gRNA. In some embodiments, the DNA-binding protein target sequence can have at least one (e.g., one, two, three, four, five, six, seven, eight, nine, or ten) mismatched nucleotide to its corresponding nucleotide in the donor gRNA when the DNA-binding protein target sequence and the donor gRNA are hybridized. Examples of mismatched bases include a guanine and uracil, guanine and thymine, and adenine and cytosine pairing. In some embodiments, the DNA-binding protein target sequence has the same sequence as a portion of the target nucleic acid.

One or more DNA-binding protein target sequences can be present on one or both termini of the HDR template in the donor template as described above. The DNA-binding protein target sequence and the PAM in a donor template can have different configurations as described above. The DNA-binding protein target sequence is only recognized and bound, but not cut, by the DNA-binding protein (e.g., an RNA-guided nuclease). In some embodiments, the DNA-binding protein target sequence is complementary to an equal length portion of the guide sequence of the donor gRNA. In some embodiments, the DNA-binding protein target sequence has at least 14 nucleotides, e.g., between 14 and 20 nucleotides (e.g., between 14 and 19, between 14 and 18, between 14 and 17, between 14 and 16, or between 14 and 15 nucleotides; 14, 15, 16, 17, 18, 19, or 20 nucleotides). A DNA-binding protein target sequence can include 12-20, 14-20, 14-19, 16-18, 15-17, 12, 13, 14, 15, 16, 17, 18, 19, or 20 nucleotides. In some embodiments, the DNA-binding protein target sequence is partially complementarity, i.e., comprises nucleotide mismatches, compared to an equal length portion of the sequence of the donor gRNA. For example, the DNA-binding protein target sequence having 20 nucleotides can have between 1 and 6 nucleotide mismatches (e.g., between 1 and 5, between 1 and 4, between 1 and 3, between 1 and 2 nucleotide mismatches; 1, 2, 3, 4, 5, or 6 nucleotide mismatches) compared to a 20-nucleotide portion of the sequence of the donor gRNA.

In some embodiments, the DNA-binding protein target sequence hybridizes to a portion of the guide sequence of the donor gRNA, e.g., the DNA-binding protein target sequence is truncated or includes mismatches compared to the guide sequence of the donor gRNA. In some cases, the DNA-binding protein target sequence has the same sequence as a portion of the target nucleic acid.

In some cases, the DNA-binding protein target sequence is a truncated CRISPR-Cas targeting (e.g., truncated CRISPR-Cas9 targeting) sequence (tCTS), wherein the DNA-binding protein target sequence has the same sequence as a portion of the target nucleic acid and hybridizes to a portion of the guide sequence of the donor gRNA. In some cases, a tCTS has 100% complementarity to a portion of the guide sequence of the guide RNA (the donor gRNA). In such cases, the tCTS is long enough to hybridize with the donor gRNA and thereby guide the CRISPR-Cas RNP to the DNA-binding protein target sequence, but is short enough such that the CRISPR-Cas effector protein (e.g., Cas9) does not cleave. As such, a tCTS allows a CRISPR-Cas RNP to bind but not cleave, even if the effector protein is a fully active nuclease. In some cases, the DNA-binding protein target sequence is 100% complementary to a portion of the guide sequence of the donor gRNA, e.g., 100% complementary to 10-16 nucleotides (nt) (e.g., 10-15, 10-14, 10-13, 10-12, 10-11, 11-16, 11-15, 11-14, 11-13, 11-12, 12-16, 12-15, 12-14, 12-13, 13-16, 13-15, or 13-14 nt). In some cases, the DNA-binding protein target sequence is 100% complementary to a portion of the guide sequence of the donor gRNA, e.g., 100% complementary to 10-15 nucleotides (nt) (e.g., 10-14, 10-13, 10-12, 10-11, 11-15, 11-14, 11-13, 11-12, 12-15, 12-14, 12-13, 13-15, or 13-14 nt). For more information related to tCTS, refer to Nguyen et al., Nat Biotechnol. 2020 January; 38(1):44-49; Nguyen et al., (2019) bioRxiv 591719; doi: https://doi.org/10.1101/591719; and to US Patent Application Publication US20220017882, all of which are incorporated herein by reference.

In some embodiments, a donor template includes one DNA-binding protein target sequence (e.g., one tCTS). In some embodiments, a donor template includes two DNA-binding protein target sequences (e.g., two tCTSs). In some embodiments, a donor template includes two or more DNA-binding protein target sequences (e.g., 3 or more, 4 or more, 5 or more, or 6 or more tCTSs). In some embodiments, a donor template includes more than two DNA-binding protein target sequences (e.g., 3, 4, 5, 6, 7, 8, 9, or 10 tCTSs).

### Lipid Nanoparticles

In some embodiments of the compositions described herein, a lipid nanoparticle can be added to a composition, e.g., to improve the stability and editing efficiency of Cas9 protein and sgRNA ribonucleoprotein complex (RNP). The present disclosure provides lipid nanoparticles for delivery of a molecular payload (e.g., a nucleic acid payload such as DNA or RNA, e.g., mRNA, and/or a protein payload, e.g., a CRISPR-Cas effector protein such as a Cas9 protein) to cells and tissues. In some cases, the payload includes a ribonucleoprotein (RNP), e.g., a CRISPR-Cas guide RNA complexed with a CRISPR-Cas effector protein. As used herein, the phrase “lipid nanoparticle” (LNP) refers to a transfer vehicle comprising one or more lipids (e.g., ionizable lipids, cationic lipids, non-cationic lipids, neutral lipids, neutral phospholipids, polymerizable lipids, PEG-modified lipids, cholesterol, and the like). In some cases, an LNP includes a molecular payload. In some embodiments, a subject LNP includes an ionizable lipid (e.g., OF-02, L-319), a neutral phospholipid (e.g., DOPE), cholesterol, and a pegylated lipid (e.g., DMG-PEG-2000). In some embodiments, the LNP includes a cationic lipid (e.g., DOTAP). In some embodiments, the LNP does not include a cationic lipid. In some embodiments, the LNP has a size of less than 125 nm. In some embodiments, the LNP has a size of less than 100 nm.

Lipid nanoparticles may be, e.g., microspheres (including unilamellar and multilamellar vesicles, e.g. “liposomes”-lamellar phase lipid bilayers that, in some embodiments are substantially spherical, and, in more particular embodiments can comprise an aqueous core, a dispersed phase in an emulsion, micelles or an internal phase in a suspension.

Methods of preparing/producing lipid nanoparticles and delivering a payload contained in a lipid nanoparticle are described in the art and include, but are not limited, those described in, for example, U.S. Pat. No. 9,737,604 and Zhang et al. “Lipid nanoparticle-mediated efficient delivery of CRISPR-Cas9 for tumor therapy,” NPG Asia Materials Volume 9, page e441 (2017).

### Ionizable Lipids

In some embodiments, a subject LNP includes an ionizable lipid. Ionizable lipids are protonated at low pH, which makes them positively charged, but they remain neutral at physiological pH. The pH-sensitivity of ionizable lipids is beneficial, e.g., for mRNA delivery in vivo, because neutral lipids have less interactions with the anionic membranes of cells and, thus, improve the biocompatibility of lipid nanoparticles. In some cases, ionizable lipids can promote endosome escape and reduce toxicity.

In some cases, an ionizable lipid of a subject LNP includes a branched tail (e.g., Lipid III-45, lipid A9, ALC-0315, and the like). Examples of ionizable lipids include, but are not necessarily limited to: OF-02, L-319, BP lipid 312, LP01, Lipid III-45, ALC-0315, lipid A9, D-Lin, DLin-MC3-DMA, ALC-0315, SM-102, LP01, CL1, TCL053, CKK-E12, ATX-002, DLin-DMA, DLenDMA, DLin-D-DMA, DLin-K-DMA, DLin-M-C2-DMA, DLin-K-DMA, DLin-KC2-DMA, DLin-KC3-DMA, DLin-KC4-DMA, DLin-C2K-DMA, DLin-MP-DMA, DODMA, 98N12-5, C12-200, DLin-C-DAP, DLin-DAC, DLinDAP, DLinAP, DLin-EG-DMA, DLin-2-DMAP, KL10, KL22, KL25, Octyl-CLinDMA, Octyl-CLinDMA (2R), Octyl-CLinDMA (2S), or an analog thereof.

In some embodiments, a subject LNP includes the ionizable lipid OF-02 or the ionizable lipid L-319. In some embodiments, a subject LNP includes the ionizable lipid OF-02. In some embodiments, a subject LNP includes the ionizable lipid L-319.

In some cases, the ionizable lipid (e.g., OF-02 or L-319) is present in a subject LNP in a mole percentage (mole %) of about 10-50% of the total lipids (e.g., 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-40, 30-35, 35-50, 35-45, 35-40, 40-50, 40-45, 45-50). In some cases, the ionizable lipid (e.g., OF-02 or L-319) is present in a subject LNP in a mole percentage (mole %) of about 15-25% of the total lipids. In some cases, the ionizable lipid (e.g., OF-02 or L-319) is present in a subject LNP in a mole percentage (mole %) of about 30-40% of the total lipids.

In some cases, the ionizable lipid OF-02 is present in a subject LNP in a mole percentage (mole %) of about 15-25% of the total lipids (e.g., 15-23, 15-20, 15-18, 15-16, 16-23, 16-20, 16-18, 16-17, 18-23, 18-20, 18-19, 20-25, 20-23, 20-22, 22-25, 24-25). In some cases, the ionizable lipid OF-02 is present in a subject LNP in a mole percentage (mole %) of about 15-20% of the total lipids (e.g., 15-20, 15-18, 15-16, 16-20, 16-18, 16-17, 18-20). In some cases, the ionizable lipid OF-02 is present in a subject LNP in a mole percentage (mole %) of about 18% of the total lipids.

### Structure of OF-02

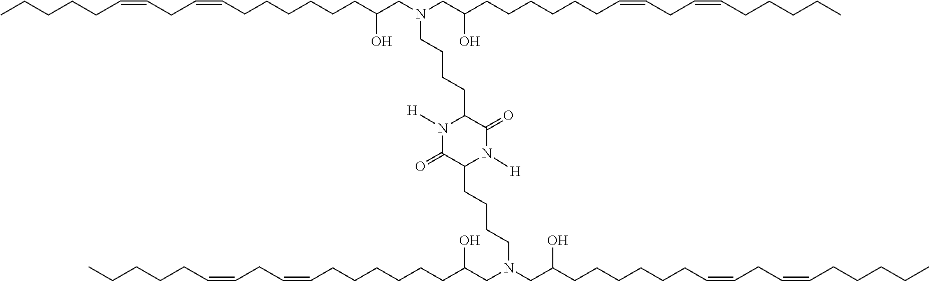 [SMILES: CCCCC/C=C\C/C=C\CCCCCCC(O)CN(CCCCC1NC(=O)C(CCCCN(CC(O)CCCCCC/C=C\C/C=C\CCCCC)CC(O)CCCCCC/C=C\C/C=C\CCCCC)NC1=O)CC(O)CCCCCC/C=C\C/C=C\CCCCC]

In some cases, the ionizable lipid L-319 is present in a subject LNP in a mole percentage (mole %) of about 30-40% of the total lipids (e.g., 30-38, 30-36, 30-34, 30-32, 32-38, 32-36, 32-34, 34-38, 34-36, 36-38, 36-39, 38-39, 38-40). In some cases, the ionizable lipid L-319 is present in a subject LNP in a mole percentage (mole %) of about 33-37% of the total lipids (e.g., 33-36, 33-35, 33-34, 34-36, 34-35, 35-36). In some cases, the ionizable lipid L-319 is present in a subject LNP in a mole percentage (mole %) of about 35% of the total lipids.

### Structure of L-319

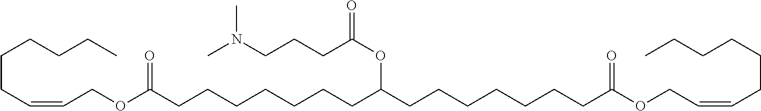 [SMILES: CCCCCC/C=C\COC(=O)CCCCCCCC(CCCCCCCC(=O)OC/C=C\CCCCCC)OC(=O)CCCN(C)C]

### Neutral Phospholipids

In some embodiments, a subject LNP includes a neutral phospholipid (e.g., DOPE). In some cases, the neutral phospholipid (e.g., DOPE) is present in a subject LNP in a mole percentage (mole %) of about 15-40% of the total lipids (e.g., 15-35, 15-30, 15-25, 15-20, 20-40, 20-35, 20-30, 20-25, 25-40, 25-35, 25-30, 30-40, 35-40). In some cases, the neutral phospholipid (e.g., DOPE) is present in a subject LNP in a mole percentage (mole %) of about 20-30% of the total lipids (e.g., 20-28, 20-26, 20-24, 20-22, 22-28, 22-26, 22-24, 24-28, 24-26, 26-28). In some cases, the neutral phospholipid (e.g., DOPE) is present in a subject LNP in a mole percentage (mole %) of about 22-26% of the total lipids (e.g., 22-25, 22-24, 22-23, 23-25, 23-24, 24-25). In some cases, the neutral phospholipid (e.g., DOPE) is present in a subject LNP in a mole percentage (mole %) of about 24% of the total lipids.

### Structure of DOPE

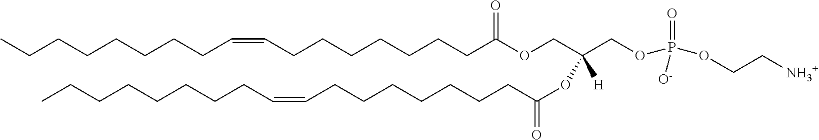 [SMILES: CCCCCCCC/C=C\CCCCCCCC(=O)OC[C@H](COP(=O)([O-])OCC[NH3+])OC(=O)CCCCCCC/C=C\CCCCCCCC]

### 1,2-Dioleoyl-sn-glycero-3-phosphoethanolamine

Examples of neutral phospholipids include, but are not necessarily limited to: 5-heptadecylbenzene-1,3-diol (resorcinol), dipalmitoylphosphatidylcholine (DPPC), distearoylphosphatidylcholine (DSPC), pohsphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In one embodiment, the neutral phospholipid may be selected from the group consisting of distearoylphosphatidylcholine (DSPC) and dimyristoyl phosphatidyl ethanolamine (DMPE). In another embodiment, the neutral phospholipid may be distearoylphosphatidylcholine (DSPC).

### Cholesterol

In some embodiments, a subject LNP includes cholesterol. In some cases, the cholesterol is present in a subject LNP in a mole percentage (mole %) of about 30-50% of the total lipids (e.g., 32-50, 32-46, 32-44, 32-42, 32-41, 34-50, 34-48, 34-46, 34-44, 34-42, 34-41, 36-50, 36-48, 36-46, 36-44, 36-42, 36-41, 38-50, 38-48, 38-46, 38-44, 38-42, 38-41, 39-50, 39-48, 39-46, 39-44, 39-42, or 39-41). In some cases, the cholesterol is present in a subject LNP in a mole percentage (mole %) of about 35-45% of the total lipids (e.g., 36-44, 36-42, 36-41, 38-45, 38-44, 38-42, 38-41, 39-45, 39-44, 39-42, or 39-41). In some cases, the cholesterol is present in a subject LNP in a mole percentage (mole %) of about 40% of the total lipids.

In some cases, the cholesterol is present in a subject LNP in a mole percentage (mole %) of about 10-25% of the total lipids (e.g., 10-23, 10-20, 10-18, 10-17, 12-25, 12-23, 12-20, 12-18, 12-17, 13-25, 13-23, 13-20, 13-18, 13-17, 14-25, 14-23, 14-20, 14-18, 14-17, 15-25, 15-23, 15-20, 15-18, 15-17, 15, or 16). In some cases, the cholesterol is present in a subject LNP in a mole percentage (mole %) of about 14-20% of the total lipids (e.g., 14-18, 14-17, 15-18, 15-17, 15, or 16). In some cases, the cholesterol is present in a subject LNP in a mole percentage (mole %) of about 15.8% of the total lipids. In some cases, the cholesterol is present in a subject LNP in a mole percentage (mole %) of about 16% of the total lipids.

### Structure of Cholesterol

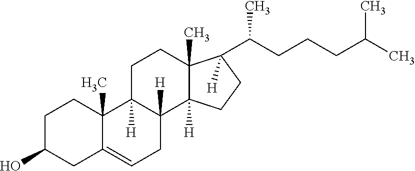

### Pegylated Cholesterol

In some embodiments, a subject LNP includes a pegylated cholesterol (e.g., Chol-PEG-2000). In some cases, the pegylated cholesterol (e.g., Chol-PEG-2000) is present in a subject LNP in a mole percentage of about 0.2-1% of the total lipids (e.g., 0.2-0.6, 0.3-0.5, or about 0.4). In some cases, the pegylated cholesterol (e.g., Chol-PEG-2000) is present in a subject LNP in a mole percentage of about 0.2-0.6% of the total lipids (e.g., 0.3-0.5, or about 0.4). In some cases, the pegylated cholesterol (e.g., Chol-PEG-2000) is present in a subject LNP in a mole percentage of about 0.3-0.5% of the total lipids. In some cases, the pegylated cholesterol (e.g., Chol-PEG-2000) is present in a subject LNP in a mole percentage of about 0.4% of the total lipids. In some embodiments, the PEG moiety has a size of about 1000, 2000, 5000, 10,000, 15,000 or 20,000 daltons.

### Structure of Chol-PEG-2000

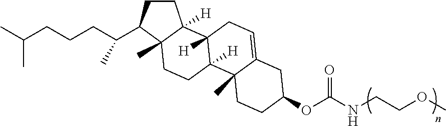

### Pegylated Lipids

In some embodiments, a subject LNP includes a pegylated lipid (e.g., DMG PEG 2000). In some cases, the pegylated lipid (e.g., DMG PEG 2000) is present in a subject LNP in a mole percentage (mole %) of about 0.5-4% of the total lipids (e.g., 0.5-3, 0.5-1.5, 0.8-2, 1-4, 1-3, 1-2, 1.2, or 1.5). In some cases, the pegylated lipid (e.g., DMG PEG 2000) is present in a subject LNP in a mole percentage (mole %) of about 1-4% of the total lipids (e.g., 1-3, 1-2). In some cases, the pegylated lipid (e.g., DMG PEG 2000) is present in a subject LNP in a mole percentage (mole %) of 1-3% of the total lipids (e.g., 1-2, 1.2, or 1.5). In some cases, the pegylated lipid (e.g., DMG PEG 2000) is present in a subject LNP in a mole percentage (mole %) of 1-2% of the total lipids (e.g., xx). In some cases, the pegylated lipid (e.g., DMG PEG 2000) is present in a subject LNP in a mole percentage (mole %) of about 1.2% of the total lipids. In some cases, the pegylated lipid (e.g., DMG PEG 2000) is present in a subject LNP in a mole percentage (mole %) of about 1.5% of the total lipids.

### Structure of DMG-PEG (e.g., DMG-PEG 2000)

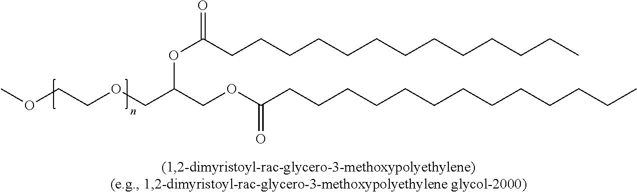

PEG-modified lipids (also referred to as pegylated lipids) (e.g., DMG-PEG 2000) include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. For example, a PEG lipid can be PEG-c-DOMG, PEG-DMG (DMG-PEG), PEG-DLPE, PEG DMPE, PEG-DPPC, or a PEG-DSPE lipid. In some embodiments, the PEG-lipid are 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA). In some embodiments, the PEG moiety has a size of about 1000, 2000, 5000, 10,000, 15,000 or 20,000 daltons. In some cases, instead of being PEG-modified, the polymerizable lipid can be modified with other hydrophilic polymers (other than PEG)—e.g., in some cases, the lipids in this paragraph can be modified with a hydrophilic polymer other than PEG.

### Cationic Lipids

Cationic lipids typically have a positively charged head group followed by a hydrophobic tail of varying composition. In an aqueous environment, these cationic lipids form micelles with positively charged surfaces that complex with DNA. Examples of cationic lipids include, but are not limited to: ADC, 1,2-dioleoyl-3-dimethylammonium-propane (DODAP), 1,2-dioleoyl-3-trimethylammonium-propane (DOTAP), N-[1-(2,3-dioleoyloxy)propyl]-N,N,N-trimethylammonium chloride (DOPTAC), 1,2-dioleoyl-3-(2-(dimethylamino)ethoxy)propylamine (DODEA), and 1,2-dimyristoyl-3-trimethylammonium-propane (DMTAP).

In some embodiments, a subject LNP includes a cationic lipid (e.g., DOTAP). In some cases, the pegylated lipid (e.g., DOTAP) is present in a subject LNP in a mole percentage (mole %) of about 10-30% (e.g., 10-28, 10-25, 10-22, 10-20, 10-18, 10-16, 10-14, 10-12, 12-30, 12-28, 12-25, 12-22, 12-20, 12-18, 12-16, 12-14, 15-30, 15-28, 15-25, 15-22, 15-20, 15-18, 15-16, 18-30, 18-28, 18-25, 18-22, 18-20, 20-30, 20-28, 20-25, 20-22, 22-30, 22-28, 22-25, 25-30, 25-26, 28-30). In some cases, the pegylated lipid (e.g., DOTAP) is present in a subject LNP in a mole percentage (mole %) of about 15-25% (e.g., 15-24, 15-22, 15-20, 15-18, 18-25, 28-22, 18-20, 20-25, 20-22, 22-25). the pegylated lipid (e.g., DOTAP) is present in a subject LNP in a mole percentage (mole %) of about 17%.

Payloads: Nucleic Acid and/or Protein

LNPs can be used as transfer vehicles for delivery of a molecular payload (e.g., nucleic acid payload such as an RNA, e.g., siRNA, mRNA, guide RNA, or DNA) into a target cell. Examples of molecular payloads that can be encapsulated by a LNP include, but are not necessarily limited to: nucleic acids (e.g., an mRNA encoding a CRISPR-Cas effector protein such as a Cas9, a donor template, a gRNA), proteins (e.g., a therapeutic protein such as an antibody, a CRISPR-Cas effector protein such as a Cas9), polysaccharides, lipids, radioactive substances, therapeutic agents, prodrugs, nutritional supplements, biomarkers, or any combination thereof. In some instances, the payload may comprise more than one type of entity such as a protein (e.g., a gene editing protein such as a Cas9) and a nucleic acid (e.g., a guide RNA).

In some cases, a molecular payload includes a nucleic acid such as an mRNA or DNA encoding a protein. In some cases, a molecular payload includes a protein, e.g., a gene editing protein. In some embodiments, a molecular payload includes a targetable nuclease. In some embodiments, a molecular payload includes a DNA-binding protein.

In some embodiments, the payload comprises a nucleic acid. Examples of such include, but are not necessarily limited to siRNAs, mRNAs, antisense oligonucleotides, miRs, anti-miRs, shRNAs, expression vectors such as plasmid DNA, DNAs or RNAs encoding a CRISPR-Cas effector protein, CRISPR-Cas guide RNAs, DNAs encoding a CRISPR-Cas guide RNA, ssDNA or dsDNAs encoding a donor template, or any combination thereof. For example, in some embodiments, the payload includes an RNA (e.g., an mRNA encoding a CRISPR-Cas effector protein). In some embodiments, the payload includes an mRNA (e.g., an mRNA encoding gene of interest, an mRNA encoding a gene editing protein such as a Zinc Finger Nuclease (ZFN), a Transcription Activator-Like Effector Nucleases (TALEN), or a CRISPR-Cas effector protein, and the like). In some embodiments, the payload includes a CRISPR-Cas guide RNA. In some embodiments, the payload includes a CRISPR-Cas guide RNA and an mRNA (e.g., an mRNA encoding a gene editing protein such as CRISPR-Cas effector protein). In some embodiments, the payload includes 2 or more nucleic acids (e.g., two or more siRNAs, two or more mRNAs, two or more guide RNAs, one or more siRNAs plus one or more guide RNAs, one or more siRNAs plus one or more mRNAs, one or more mRNAs plus one or more guide RNAs, one or more siRNAs plus one or more mRNAs plus one or more guide RNA, and the like).

In some embodiments, the payload includes a CRISPR-Cas effector protein (e.g., Cas9). In some embodiments, the payload includes a CRISPR-Cas effector protein (e.g., Cas9) and a CRISPR-Cas guide RNA (e.g., Cas9 guide RNA), e.g., in some cases complexed to one another thus forming a ribonucleoprotein (RNP). In some cases, the payload includes a CRISPR-Cas guide RNA (e.g., a Cas9 guide RNA) and an mRNA encoding a CRISPR-Cas effector protein (e.g., a Cas9). In some cases, the payload includes a donor template. In some embodiments, the payload includes a CRISPR-Cas effector protein and a donor template.

In some embodiments, the payload of an LNP includes a CRISPR-Cas effector protein (e.g., Cas9) and a CRISPR-Cas guide RNA (e.g., a Cas9 guide RNA) complexed to one another as to form a RNP, and a donor template (e.g., having one or more tCTSs, e.g., a tCTS in the region 5′ of the HDR template and a tCTS in the region 3′ of the HDR template, each of which is adjacent a PAM sequence). In some cases the donor template is complexed with the RNP.

In some embodiments, the payload of an LNP includes mRNA encoding a CRISPR-Cas effector protein (e.g., Cas9), a CRISPR-Cas guide RNA (e.g., a Cas9 guide RNA), and a donor template (e.g., having one or more tCTS, e.g., a tCTS in the region 5′ of the HDR template and a tCTS in the region 3′ of the HDR template, each of which is adjacent a PAM sequence), e.g., in some cases complexed with the RNP.

In some cases, two or more molecular payloads are delivered as part of the same LNP. In some cases, two or more molecular payloads are delivered as part of separate (different) LNPs—and can be delivered simultaneously (as part of the same or different compositions), or delivered serially (one before the other).

In some embodiments, the LNP and payload has a total lipid to nucleic acid weight ratio is in a range of from about 40:1 to 1:40 (e.g., from about 40:2, 40:5, 40:8, 40:10, 40:12, 40:15, 40:18, 40:20, 40:25, 40:30, 40:35, 35:1, 35:2, 35:5, 35:8, 35:10, 35:12, 35:15, 35:18, 35:20, 35:25, 35:30, 35:40, 30:1, 30:2, 30:5, 30:8, 30:10, 30:12, 30:15, 30:18, 30:20, 30:25, 30:35, 30:40, 25:1, 25:2, 25:5, 25:8, 25:10, 25:12, 25:15, 25:18, 25:20, 25:30, 25:35, 25:40, 20:1, 20:2, 20:5, 20:8, 20:10, 20:12, 20:15, 20:18, 20:25, 20:30, 20:35, 20:40, 18:1, 18:2, 18:5, 18:8, 18:10, 18:12, 18:15, 18:20, 18:25, 18:30, 18:35, 18:40, 15:1, 15:2, 15:5, 15:8, 15:10, 15:12, 15:18, 15:20, 15:25, 15:30, 15:35, 15:40, 12:1, 12:2, 12:5, 12:8, 12:10, 12:15, 12:18, 12:20, 12:25, 12:30, 12:35, 12:40, 10:1, 10:2, 10:5, 10:8, 10:12, 10:15, 10:18, 10:20, 10:25, 10:30, 10:35, 10:40, 8:1, 8:2, 8:5, 8:10, 8:12, 8:15, 8:18, 8:20, 8:25, 8:30, 8:35, 8:40, 5:1, 5:2, 5:8, 5:10, 5:12, 5:15, 5:18, 5:20, 5:25, 5:30, 5:35, 5:40, 2:1, 2:5, 2:8, 2:10, 2:12, 2:15, 2:18, 2:20, 2:25, 2:30, 2:35, 2:40, 1:1, 1:2, 1:5, 1:8, 1:10, 1:12, 1:15, 1:18, 1:20, 1:25, 1:30, 1:35, 1:40).

### Lyophilized Composition

A lipid nanoparticle can be used to stabilize a composition described herein during lyophilization and preserve protein activity after the lyophilized composition was reconstituted. In some embodiments, if the composition containing the Cas protein (e.g., Cas9 protein) and the lipid nanoparticle or the composition containing the Cas protein (e.g., Cas9 protein) and sgRNA RNP complexes and the lipid nanoparticle is lyophilized and later reconstituted, the Cas protein has activity for at least one week (e.g., at least 2, 3, 4, 5, 6, 7, 8, 9, 10, 11, 12, 13, 14, 15, 16, 17, 18, 19, 20, 21, 22, 23, or 24 weeks) at room temperature, 4° C., or 37° C. after reconstitution. In some embodiments, if a composition is lyophilized and later reconstituted, the targetable nuclease and/or the DNA-binding protein in the composition has activity for at least one week (e.g., at least 2, 3, 4, 5, 6, 7, 8, 9, 10, 11, 12, 13, 14, 15, 16, 17, 18, 19, 20, 21, 22, 23, or 24 weeks) at room temperature, 4° C., or 37° C. after reconstitution.

In some embodiments, a cryoprotectant can be added to a composition described herein before the composition is lyophilized. Examples of cryoprotectants include, but are not limited to, dimethyl sulfoxide (DMSO), glycerol, ethylene glycol, propylene glycol, 2-Methyl-2,4-pentanediol (MPD), sucrose, lactose, trehalose, raffinose, mannitol, and combinations thereof. Cryoprotectants added to the composition described herein to form a lyophilized composition does not affect the activity of the RNP complexes.

### RNP Complex Formation in the Presence of Lipid Nanoparticles

In some embodiments, an RNP complex may be formed by annealing an sgRNA to a Cas protein, as described in, e.g., Schumann et al. PNAS 112: 10437-10442, 2015 and Hultquist et al. Cell Rep. 17: 1438-1452, 2016, in the presence of lipid nanoparticle. In some embodiments, the RNP complex may be further complexed with the donor template. In other embodiments, a lipid nanoparticle may be added to a composition comprising an sgRNA and a Cas protein, and optionally a donor template. In other words, to form an RNP complex, in some embodiments, a Cas protein and an sgRNA, and optionally a donor template, may be incubated together first, i.e., incubated at between 25° C. and 37° C. (e.g., at 25° C., 27° C., 29° C., 31° C., 33° C., or 35° C.; at 25° C. or at 37° C.) for at least 15 minutes (e.g., 20, 25, 30, 35, 40, 45, 50, 55, 60, 65, 70, 75, 80, 85, 90, 95, 100, 110, or 120 minutes), followed by the addition of a lipid nanoparticle. Once the lipid nanoparticle is added, the mixture containing all the components (the Cas protein, the sgRNA, and the lipid nanoparticle, and optionally the donor template) may be further incubated at between 25° C. and 37° C. (e.g., at 25° C., 27° C., 29° C., 31° C., 33° C., or 35° C.; at 25° C. or at 37° C.) for at least 15 minutes (e.g., 20, 25, 30, 35, 40, 45, 50, 55, 60, 65, 70, 75, 80, 85, 90, 95, 100, 110, or 120 minutes). In other embodiments, all the components (the Cas protein, the sgRNA, and the lipid nanoparticle, and optionally the donor template) may be added together in a mixture at approximately the same time and incubated together at between 25° C. and 37° C. (e.g., at 25° C., 27° C., 29° C., 31° C., 33° C., or 35° C.; at 25° C. or at 37° C.) for at least 15 minutes (e.g., 20, 25, 30, 35, 40, 45, 50, 55, 60, 65, 70, 75, 80, 85, 90, 95, 100, 110, or 120 minutes). In yet other embodiments, an sgRNA may be added to a composition containing a Cas protein and lipid nanoparticle. In some cases, a donor template may also be added to the composition. All the components (the Cas protein, the sgRNA, and the lipid nanoparticle, and optionally the donor template) may be incubated together at between 25° C. and 37° C. (e.g., at 25° C., 27° C., 29° C., 31° C., 33° C., or 35° C.; at 25° C. or at 37° C.) for at least 15 minutes (e.g., 20, 25, 30, 35, 40, 45, 50, 55, 60, 65, 70, 75, 80, 85, 90, 95, 100, 110, or 120 minutes).

In some embodiments, in a mixture comprising the components as described above (the Cas protein, the sgRNA, and the lipid nanoparticle, and optionally the donor template), the molar ratio of sgRNA:Cas protein may be between 0.25:1 and 4:1, e.g., 0.25:1, 0.5:1, 1:1, 1.2:1, 1.4:1, 1.6:1, 1.8:1, 2:1, 2.2:1, 2.4:1, 2.6:1, 2.8:1, 3:1, 3.2:1, 3.4:1, 3.6:1, 3.8:1, or 4:1. In some embodiments, in a mixture comprising the components as described above (the Cas protein, the sgRNA, and the lipid nanoparticle, and optionally the donor template) or a mixture comprising the Cas protein and the lipid nanoparticle, and optionally the donor template, the molar ratio of lipid nanoparticle:Cas protein may be, e.g., between 10:1 and 120:1, e.g., 10:1, 20:1, 30:1, 40:1, 50:1, 60:1, 70:1, 80:1, 90:1, 100:1, 110:1, or, 120:1; between 10:1 and 110:1, between 10:1 and 100:1, between 10:1 and 90:1, between 10:1 and 80:1, between 10:1 and 70:1, between 10:1 and 60:1, between 10:1 and 50:1, between 10:1 and 40:1, between 10:1 and 30:1, between 10:1 and 20:1, between 20:1 and 120:1, between 30:1 and 120:1, between 40:1 and 120:1, between 50:1 and 120:1, between 60:1 and 120:1, between 70:1 and 120:1, between 80:1 and 120:1, between 90:1 and 120:1, between 100:1 and 120:1, or between 110:1 and 120:1. In some embodiments, the RNP complexes are electroporated into cells immediately after formation. In other embodiments, a Cas protein and lipid nanoparticle may be electroporated into cells and an sgRNA and/or the donor template may be introduced into cells via viral delivery. Methods of forming RNP complexes are also described in, e.g., International Patent Application No. PCT/US2018/037919 and U.S. Patent Application Nos. 62/676,650 and 62/578,153.

In some embodiments, forming an RNP complex containing a Cas protein (e.g., a Cas9 protein) and an sgRNA, and optionally a donor template, in the presence of a lipid nanoparticle may include adding an anionic polymer. Thus, in some embodiments, a subject LNP includes an anionic polymer. In some embodiments, the anionic polymer is an anionic polypeptide or an anionic polysaccharide. In some embodiments, the anionic polymer is an anionic polypeptide (e.g., a polyglutamic acid (PGA), a polyaspartic acid, or polycarboxyglutamic acid). In some embodiments, the anionic polymer is an anionic polysaccharide (e.g., hyaluronic acid (HA), heparin, heparin sulfate, or glycosaminoglycan). In some embodiments, the anionic polymer is poly(acrylic acid) (PAA), poly(methacrylic acid) (PMAA), poly(styrene sulfonate), or polyphosphate.

In some embodiments, the anionic polymer is added to a guide RNA (e.g., sgRNA) before a Cas protein (e.g., a Cas9 protein), and optionally a donor template, is added. In some embodiments, the volume ratio of the anionic polymer:sgRNA may be between 0.1:1.5 and 1.5:0.1, e.g., 0.1:1.5, 0.5:1.5, 0.8:1.5, 1.0:1.5, 1.2:1.5, 1.4:1.5, 0.1:1.4, 0.5:1.4, 0.8:1.4, 1.0:1.4, 1.2:1.4, 1.5:1.4, 0.1:1.2, 0.5:1.2, 0.8:1.2, 1.0:1.2, 1.4:1.2, 1.5:1.2, 0.1:1.0, 0.5:1.0, 0.8:1.0, 1.2:1.0, 1.4:1.0, 1.5:1.0, 0.1:0.5, 0.8:0.5, 1.0:0.5, 1.2:0.5, 1.4:0.5, 1.5:0.5, 0.5:0.1, 0.8:0.1, 1.0:0.1, 1.2:0.1, 1.4:0.1, 1.5:0.1. In some cases, the anionic polymer is PGA and the volume ratio of PGA:sgRNA is about 0.8:1.0.

In some embodiments, an RNP complex containing a Cas protein (e.g., a Cas9 protein) and an sgRNA, and optionally a donor template, that is formed in the presence of a lipid nanoparticle has a size that is less than 100 nm (e.g., 95, 90, 85, 80, 75, 70, 65, 60, 55, 50, 45, 40, 35, 30, 25, or 20 nm). A Cas protein and sgRNA RNP complex, or the RNP complex further complexed with the donor template, may have a size that is between 20 nm and 90 nm (e.g., 20, 25, 30, 35, 40, 45, 50, 55, 60, 65, 70, 75, 80, 85, or 90 nm), as determined by known techniques in the art, e.g., dynamic light scattering. In some embodiments, a composition containing a Cas protein and sgRNA RNP complex and lipid nanoparticle, and optionally the donor template, includes at least 50% (e.g., 55%, 60%, 65%, 70%, 75%, 80%, 85%, 90%, 95%, or 100%) of the RNP complexes that have a size that is between 20 nm and 90 nm (e.g., 20, 25, 30, 35, 40, 45, 50, 55, 60, 65, 70, 75, 80, 85, or 90 nm).

### Gene Targeting Nucleic Acids in Cells

The compositions described herein can be used in methods of modifying a target nucleic acid in a cell, e.g., a eukaryotic cell, prokaryotic cell, animal cell, plant cell, fungal cell, and the like. Optionally, the cell is a mammalian cell, for example, a human cell. The cell can be in vitro, ex vivo, or in vivo. The cell can also be a primary cell, a germ cell, a stem cell, or a precursor cell. The precursor cell can be, for example, a pluripotent stem cell, or a hematopoietic stem cell. In some embodiments, the cell is a primary hematopoietic cell, a primary hematopoietic stem cell, or a primary T cell. In some embodiments, the primary hematopoietic cell is an immune cell. In some embodiments, the immune cell is a T cell. In some embodiments, the T cell is a regulatory T cell, an effector T cell, or a naïve T cell. In some embodiments, the T cell is a CD4⁺ T cell. In some embodiments, the T cell is a CD8⁺ T cell. In some embodiments, the T cell is a CD4⁺CD8⁺ T cell. In some embodiments, the T cell is a CD4⁻CD8⁻ T cell. In some embodiments the T cell is an as T cell, in some embodiments the T cell is a γδ T cell. Populations of any of the cells modified by any of the methods described herein are also provided. In some embodiments, the methods further comprise expanding the population of modified cells.

In a particular aspect, a population of cells (e.g., a population of T cells), is provided. The population of cells can comprise any of the modified cells described herein. The modified cell can be within a heterogeneous population of cells and/or a heterogeneous population of different cell types. The population of cells can be heterogeneous with respect to the percentage of cells that are genomically edited. A population of cells can have greater than 10%, greater than 20%, greater than 30%, greater than 40%, greater than 50%, greater than 60%), greater than 70%, greater than 80%, or greater than 90% of the population comprise an integrated nucleotide sequence. In a certain aspect, a populations of cells comprises an integrated nucleotide sequence, wherein the integrated nucleotide sequence comprises at least a portion of a gene, the integrated nucleotide sequence is integrated at an endogenous genomic target locus, and the integrated nucleotide sequence is orientated such that the at least a portion of the gene is capable of being expressed, wherein the population of cells is substantially free of viral-mediated delivery components, and wherein greater than 10%), greater than 20%, greater than 30%, greater than 40%, greater than 50%, greater than 60%), greater than 70%, greater than 80%, or greater than 90% of the cells in the population comprise the integrated nucleotide sequence.

A population of cells can have greater than 91%, greater than 92%, greater than 93%), greater than 94%, greater than 95%, greater than 96%, or greater than 97%, greater than 98%), greater than 99%, greater than 99.5%, or greater than 99.9% of the population comprise an integrated nucleotide sequence. A population of cells can have greater than 20% of the population comprise an integrated nucleotide sequence. A population of cells can have greater than 30% of the population comprise an integrated nucleotide sequence. A population of cells can have greater than 60% of the population comprise an integrated nucleotide sequence. A population of cells can have greater than 70% of the population comprise an integrated nucleotide sequence.

A cell can include a cell comprising a non-virally inserted sequence such as an exogenous sequence. A cell can be virus-free. A cell can be substantially free of virus. A cell can include at least one nucleic acid sequence (e.g., comprising at least one heterologous gene) non-virally inserted into at least one target region. In certain aspects, a cell does not comprise a viral vector, e.g., for introducing at least one nucleic acid sequence such as a donor template.

A cell can include one or more primary cells that include a non-virally inserted exogenous sequence that is at least 200 base pairs in size. The primary cells can be primary hematopoietic cells or primary hematopoietic stem cells. The primary cells can be primary hematopoietic cells and the primary hematopoietic cells can be immune cells. The immune cells can be T cells. The primary cells can be human cells. In some aspects, the primary cells do not comprise a viral vector. The size of the exogenous sequence can be greater than a length selected from the group consisting of: 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 1 kb, 1.1 kb, 1.2 kb, 1.3 kb, 1.4 kb, 1.5 kb, 1.6 kb, 1.7 kb, 1.8 kb, 1.9 kb, 2.0 kb, 2.1 kb, 2.2 kb, 2.3 kb, 2.4 kb, 2.5 kb, 2.6 kb, 2.7 kb, 2.8 kb, 2.9 kb, 3 kb, 3.1 kb, 3.2 kb, 3.3 kb, 3.4 kb, 3.5 kb, 3.6 kb, 3.7 kb, 3.8 kb, 3.9 kb, 4.0 kb, 4.1 kb, 4.2 kb, 4.3 kb, 4.4 kb, 4.5 kb, 4.6 kb, 4.7 kb, 4.8 kb, 4.9 kb, and 5.0 kb. The size of the exogenous sequence can be greater than 1.5 kb. The size of the exogenous sequence can be about 200 bp to about 500 bp, about 200 bp to about 750 bp, about 200 bp to about 1 kb, about 200 bp to about 1.5 kb, about 200 bp to about 2.0 kb, about 200 bp to about 2.5 kb, about 200 bp to about 3.0 kb, about 200 bp to about 3.5 kb, about 200 bp to about 4.0 kb, about 200 bp to about 4.5 kb, about 200 bp to about 5.0 kb. The size of the exogenous sequence can be greater than about 1 kb. The exogenous sequence can include a regulatory sequence, optionally wherein the regulatory sequence comprises a promoter sequence and/or an enhancer sequence.

The exogenous sequence can encode a heterologous protein or a fragment thereof. The exogenous sequence can encode a chimeric antigen receptor (CAR). As such, the cell can be a CAR-T cell. The exogenous sequence can encode a T cell receptor (TCR). As such, the cell can be a TCR-T cell.

A cell can include a primary human T cell comprising a non-virally inserted DNA template having a size of greater than 1 kb.

A cell can include a primary human T cell comprising: at least one nucleic acid sequence comprising at least one heterologous gene non-virally inserted into at least one target region of one or both of: endogenous T cell receptor alpha subunit constant gene (TRAC), and endogenous T cell receptor beta subunit constant gene (TRBC), the at least one heterologous gene comprises at least one of: (1) a variable region of a heterologous T cell receptor alpha (TCR-α) chain gene and (2) a variable region of a heterologous T cell receptor beta (TCR-β) chain gene. In some aspects, the T cell does not comprise a viral vector for introducing the at least one nucleic acid sequence to the T cell. In some aspects, the at least one nucleic acid sequence is at least 1.5 kb in size. In some aspects, the at least one nucleic acid sequence is at least 500 bp in size. In some aspects, the target region is in exon 1, 2, or 3 of TRAC. In some aspects, the target region is in exon 1, 2, or 3 of TRBC. In some aspects, the T cell is a CD8+ T cell or a CD4+ T cell. In some aspects, the at least one heterologous gene comprises at least one of: (1) a) variable region or b) variable region and constant region of the heterologous T cell receptor alpha (TCR-α) chain gene and (2) a) variable region or b) variable region and constant region of the heterologous T cell receptor beta (TCR-β) chain gene. In some aspects, the at least one heterologous gene comprises each of: (1) the a) variable region or b) variable region and constant region of the heterologous T cell receptor alpha (TCR-α) chain gene and (2) the a) variable region or b) variable region and constant region of the heterologous T cell receptor beta (TCR-β) chain gene. In some aspects, the T cell comprises each of (1) the a) variable region or b) variable region and constant region of the heterologous TCR-α chain gene and (2) the a) variable region or b) variable region and constant region of the heterologous TCR-β chain gene. In some aspects, the heterologous genes form an antigen-specific T cell receptor (TCR) upon expression. In some aspects, the heterologous TCR-α chain gene and the heterologous TCR-α chain gene are operably linked by a linker sequence, optionally the linker sequence is a cleavable linker sequence or a multicistronic element. In some aspects, the heterologous TCR-α chain gene and the heterologous TCR-β chain gene are inserted into TRAC. In some aspects, expression of the at least one heterologous gene is driven by an endogenous promoter. In some aspects, expression of one or both of TRAC and TRBC is reduced in the cell relative to a control T cell, wherein the control T cell is a primary human T cell that lacks the non-viral insertion.

A cell can include a primary human T cell comprising: at least one nucleic acid sequence comprising at least one heterologous gene inserted into at least one target region of one or both of: endogenous T cell receptor alpha subunit constant gene (TRAC), and endogenous T cell receptor beta subunit constant gene (TRBC), the at least one heterologous gene comprises at least one of: (1) a variable region of a heterologous T cell receptor alpha (TCR-α) chain gene and (2) a variable region of a heterologous T cell receptor beta (TCR-β) chain gene, and wherein the T cell does not comprise a viral vector for introducing the at least one nucleic acid sequence to the T cell.

A cell can include a primary cell comprising: at least one nucleic acid sequence comprising at least one heterologous gene non-virally inserted into at least one target region of the cell's genome. In some aspects the at least one heterologous gene encodes a CAR or other chimeric receptor. In some aspects the at least one heterologous gene comprises at least one or both of: (1) a variable region of a heterologous T cell receptor alpha (TCR-α) chain gene and (2) a variable region of a heterologous T cell receptor beta (TCR-β) chain gene. In some aspects the primary cell is a T cell. In some aspects, the cell does not comprise a viral vector for introducing the at least one nucleic acid sequence to the cell. In some aspects, the at least one nucleic acid sequence is at least 1.5 kb in size. In some aspects, the at least one nucleic acid sequence is at least 500 bp in size. In some aspects, the target region is in TRAC, e.g., exon 1, 2, or 3 of TRAC. In some aspects, the target region is in TRBC, e.g., exon 1, 2, or 3 of TRBC. In some aspects, the T cell is a CD8+ T cell or a CD4+ T cell. In some aspects, expression of the at least one heterologous gene is driven by an endogenous promoter. In some aspects, expression of one or both of TRAC and TRBC is reduced in the T cell relative to a control T cell, wherein the control T cell is a primary human T cell that lacks the non-viral insertion.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR in some aspects is one that includes multiple costimulatory domains of different costimulatory receptors. In some embodiments, the chimeric antigen receptor includes an extracellular portion containing an antibody or fragment described herein. In some aspects, the chimeric antigen receptor includes an extracellular portion containing an antibody or fragment described herein and an intracellular signaling domain. In some embodiments, an antibody or fragment includes an scFv or a single-domain VH antibody and the intracellular domain contains an ITAM. In some aspects, the intracellular signaling domain includes a signaling domain of a ζ chain of a CD3 (CD3ζ chain) In some embodiments, the chimeric antigen receptor includes a transmembrane domain linking the extracellular domain and the intracellular signaling domain. In some aspects, the transmembrane domain contains a transmembrane portion of CD28. The extracellular domain and transmembrane can be linked directly or indirectly. In some embodiments, the extracellular domain and transmembrane are linked by a spacer, such as any described herein. In some embodiments, the chimeric antigen receptor contains an intracellular domain of a T cell costimulatory molecule, such as between the transmembrane domain and intracellular signaling domain. In some aspects, the T cell costimulatory molecule is CD28 or 411BB. In some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1 BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g., an IgG4 hinge, such as a hinge-only spacer. In some embodiments, the transmembrane domain of the receptor, e.g., the CAR, is a transmembrane domain of human CD28 or variant thereof, e.g., a 27-amino acid transmembrane domain of a human CD28 (Accession No.: P10747.1). In some embodiments, the chimeric antigen receptor contains an intracellular domain of a T cell costimulatory molecule. In some aspects, the T cell costimulatory molecule is CD28 or 411BB. In some embodiments, the intracellular signaling domain comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular domain comprises an intracellular costimulatory signaling domain of 41 BB or functional variant or portion thereof, such as a 42-amino acid cytoplasmic domain of a human 4-1BB (Accession No. 007011.1) or functional variant or portion thereof. In some embodiments, the intracellular signaling domain comprises a human CD3 zeta stimulatory signaling domain or functional variant thereof, such as an 112 AA cytoplasmic domain of isoform 3 of human CD3ζ (Accession No.: P20963.2) or a CD3ζ signaling domain as described in U.S. Pat. No. 7,446,190 or 8,911,993.

Methods for modifying a target nucleic acid in a cell described herein comprise introducing into the cell a composition described herein, wherein the HDR template is integrated into the target nucleic acid. In some cases, pre-incubation of the DNA-binding protein (e.g., an RNA guided nuclease) and donor gRNA RNP complex with the donor template (comprising DNA-binding protein target sequence-modified HDR template) before introducing the composition into the cell enhances genome targeting efficiency. In some embodiments, a composition described herein is introduced into the cell via electroporation.

In some cases, the cells are removed from a subject, modified using any of the methods described herein and administered to the subject. In other cases, a composition described herein can be delivered to the subject in vivo.

In particular embodiments, the compositions described herein can be used in methods of modifying a target nucleic acid in a primary cell. The compositions described herein can be used in methods for inducing a stable gene modification of a target nucleic acid in a primary cell. In some embodiments, the method includes introducing into the primary cell a composition comprising a Cas protein or an mRNA encoding a Cas protein (e.g., a Cas9 protein), one or more single guide RNAs (sgRNAs), and a lipid nanoparticle, and optionally a donor template. The sgRNA may comprise a first nucleotide sequence that is complementary to the target nucleic acid and a second nucleotide sequence that interacts with the Cas protein (e.g., Cas9 protein). In some embodiments, a Cas protein (e.g., a Cas9 protein), an sgRNA, and a lipid nanoparticle may be incubated together to form a RNP complex prior to introducing into the primary cell. In some cases, the RNP complex is incubated with a donor template. A composition comprising a Cas protein or an mRNA encoding a Cas protein (e.g., a Cas9 protein), one or more single guide RNAs (sgRNAs), and a lipid nanoparticle, and optionally a donor template, may be electroporated into the primary cell. In other embodiments, a composition comprising a Cas protein or mRNA encoding a Cas protein and a lipid nanoparticle, and optionally a donor template, may be electroporated into the primary cell and an sgRNA may be introduced into the primary cell via viral delivery. In some embodiments, the primary cell is selected from the group consisting of an immune cell (e.g., a primary T cell), a blood cell, a progenitor or stem cell thereof, a mesenchymal cell, and a combination thereof. In some instances, the immune cell is selected from the group consisting of a T cell, a B cell, a dendritic cell, a natural killer cell, a macrophage, a neutrophil, an eosinophil, a basophil, a mast cell, a precursor thereof, and a combination thereof. The progenitor or stem cell can be selected from the group consisting of a hematopoietic progenitor cell, a hematopoietic stem cell, and a combination thereof. In some cases, the blood cell is a blood stem cell. In some instances, the mesenchymal cell is selected from the group consisting of a mesenchymal stem cell, a mesenchymal progenitor cell, a mesenchymal precursor cell, a differentiated mesenchymal cell, and a combination thereof. The differentiated mesenchymal cell can be selected from the group consisting of a bone cell, a cartilage cell, a muscle cell, an adipose cell, a stromal cell, a fibroblast, a dermal cell, and a combination thereof. In some embodiments, the primary cell can comprise a population of primary cells. In some cases, the population of primary cells comprises a heterogeneous population of primary cells. In other cases, the population of primary cells comprises a homogeneous population of primary cells.

In some embodiments, the primary cell is isolated from a mammal prior to introducing a composition described herein into the primary cell. For instance, the primary cell can be harvested from a human subject. In some instances, the primary cell or a progeny thereof is returned to the mammal after introducing the composition described herein into the primary cell. In other words, the genetically modified primary cell undergoes autologous transplantation. In other instances, the genetically modified primary cell undergoes allogeneic transplantation. For example, a primary cell that has not undergone stable gene modification is isolated from a donor subject, and then the genetically modified primary cell is transplanted into a recipient subject who is different than the donor subject.

A composition described herein can be introduced into a cell (e.g., a primary cell) using available methods and techniques in the art. Non-limiting examples of suitable methods include electroporation, particle gun technology, and direct microinjection. In some embodiments, the step of introducing the composition described herein into the cell comprises electroporating the composition into the cell.

In other embodiments, a composition comprising a Cas protein or an mRNA encoding a Cas protein and lipid nanoparticle, and optionally a donor template, may be electroporated into the cell and an sgRNA may be introduced into the cell via viral delivery using a viral vector. For example, viral vectors can be based on vaccinia virus, poliovirus, adenovirus, adeno-associated virus, SV40, herpes simplex virus, human immunodeficiency virus, and the like. A retroviral vector can be based on Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, mammary tumor virus, and the like. An sgRNA may also be introduced into the cell using other expression vectors. Useful expression vectors are known to those of skill in the art, and many are commercially available. The following vectors are provided by way of example for eukaryotic host cells: pXT1, pSG5, pSVK3, pBPV, pMSG, and pSVLSV40. Examples of techniques that may be used to introduce an sgRNA in an expression vector (e.g., a viral vector) into a cell include, but not limited to, viral or bacteriophage infection, transfection, protoplast fusion, lipofection, calcium phosphate precipitation, polyethyleneimine (PEI)-mediated transfection, DEAE-dextran mediated transfection, liposome-mediated transfection, calcium phosphate precipitation, nanoparticle-mediated nucleic acid delivery, and the like.

In some embodiments, the stable gene modification of the target nucleic acid is induced in greater than about 5% of the population of cells (e.g., the population of primary cells), e.g., about 6%, about 7%, about 8%, about 9%, about 10%, about 12%, about 14%, about 16%, about 18%, about 20%, about 22%, about 24%, about 26%, about 28%, or about 30% of the population of cells. In some embodiments, the stable gene modification of the target nucleic acid is induced in greater than about 50% of the population of cells (e.g., the population of primary cells), e.g., about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of the population of cells. In other embodiments, the stable gene modification of the target nucleic acid is induced in greater than about 70% of the population of cells, e.g., about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of the population of cells. In yet other embodiments, the stable gene modification of the target nucleic acid is induced in greater than about 90% of the population of cells, e.g., about 90%, about 91%, about 92%, about 93%, about 94%, about 95%, about 96%, about 97%, about 98%, about 99%, or about 100% of the population of cells.

In other embodiments, the stable gene modification of the target nucleic acid comprises the replacement of a genetic mutation in the target nucleic acid (e.g., to correct a point mutation or a single nucleotide polymorphism (SNP) in the target nucleic acid that is associated with a disease) or the insertion of an open reading frame (ORF) comprising a normal copy of the target nucleic acid (e.g., to knock in a wild-type cDNA of the target nucleic acid that is associated with a disease).

In some embodiments, any of the methods described herein can also include purifying the cell (e.g., a primary cell) having the stable gene modification of the target nucleic acid. In some cases, the composition isolated by the purifying step includes at least about 80% cells having the stable gene modification of the target nucleic acid, e.g., about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more cells having the stable gene modification of the target nucleic acid.

### Treatment/Administration

In some embodiments, the present disclosure provides a method of treating a disease, the method comprising administering to an individual in need thereof an effective amount of a subject LNP (comprising an appropriate molecular payload).

In some cases, LNPs can be administered systemically, regionally or locally, or by any convenient route, for example, by injection, infusion, topically (e.g., transdermally), etc. Possible delivery and administration methods can include parenteral, intravenous, intramuscular, intraperitoneal, intradermal, subcutaneous, intracavity, intracranial, transdermal (topical), transmucosal and rectal administration. Example administration and delivery routes include intravenous, intraperitoneal, intrarterial, parenteral, subcutaneous, intra-pleural, topical, dermal, intradermal, transdermal, transmucosal, oral (alimentary), mucosal, respiration, intranasal, intubation, intrapulmonary, intrapulmonary instillation, buccal, sublingual, intravascular, intrathecal, intracavity, iontophoretic, intraocular, ophthalmic, optical, intraglandular, intraorgan, and intralymphatic. In some cases the delivery route is systemic (e.g., parenteral, intravenous). In some cases, the delivery route is subcutaneous.

In some cases, a therapeutically effective amount of an LNP is an amount that, when administered to an individual in one or more doses, is effective to slow the progression of a disease or disorder in the individual, or is effective to ameliorate symptoms, or is effective to induce a desirable outcome. For example, a therapeutically effective amount of an LNP can be an amount that, when administered to an individual in one or more doses, is effective to reduce a metric of a symptom of interest, e.g., by at least about 5%, at least about 10%, at least about 15%, at least about 20%, or at least about 25%.

A therapeutic or beneficial effect of treatment is therefore any objective or subjective measurable or detectable improvement or benefit provided to a particular subject. A therapeutic or beneficial effect can but need not be complete ablation of all or any particular adverse symptom, disorder, illness, or complication of a disease. Thus, a satisfactory clinical endpoint is achieved when there is an incremental improvement or a partial reduction in an adverse symptom, disorder, illness, or complication caused by or associated with a disease, or an inhibition, decrease, reduction, suppression, prevention, limit or control of worsening or progression of one or more adverse symptoms, disorders, illnesses, or complications caused by or associated with the disease, over a short or long duration (hours, days, weeks, months, etc.).

In some cases, a subject method can include steps of evaluating the effectiveness of administration—such evaluation can include, e.g., measuring body weight, oxygen consumption rate, body composition, glucose and fatty acid metabolism parameters, shivering, and any combination thereof.

Multiple doses of a subject LNP can be administered to an individual in need thereof. Where multiple doses are administered over a period of time, an active agent can be administered once a day, once every other day, once every 3 days, once a week, once every two weeks, once a month, once every 2 months, etc., as needed/desired. The actual frequency of administration, and the actual duration of treatment, depends on various factors. The duration of administration can also vary. As an illustrative example, in some cases, administration can be once a week for 5 weeks, 10 weeks, 15 weeks, 20 weeks, etc.

The dose to achieve a therapeutic effect, e.g., the dose of molecular payload per kilogram of body weight (mg/kg), will vary based on several factors including, but not limited to: route of administration, the nature of the payload, the amount of payload required to achieve a therapeutic effect, the desired outcome, the specific disease treated, any host immune response to the treatment, the stability of the payload, and the like. One skilled in the art can readily determine an appropriate dose range for administration to an individual. For example, if the payload is an siRNA, the dose will likely range from about 0.1-2 mg/per kilogram (mg/kg) of the weight of the subject (e.g., in some cases about 0.2 mg/kg, 0.3 mg/kg, 0.4 mg/kg, 0.5 mg/kg), to achieve a therapeutic effect.

An effective amount or a sufficient amount can, but need not be, provided in a single administration, may require multiple administrations, and, can but need not be, administered alone or in combination with another composition (e.g., agent), treatment, protocol or therapeutic regimen. For example, the amount may be proportionally increased as indicated by the need of the subject, type, status and severity of the disease treated or side effects (if any) of treatment. In addition, an effective amount or a sufficient amount need not be effective or sufficient if given in single or multiple doses without a second composition (e.g., another drug or agent), treatment, protocol or therapeutic regimen, since additional doses, amounts or duration above and beyond such doses, or additional compositions (e.g., drugs or agents), treatments, protocols or therapeutic regimens may be included in order to be considered effective or sufficient in a given subject. Amounts considered effective also include amounts that result in a reduction of the use of another treatment, therapeutic regimen or protocol.

An effective amount or a sufficient amount need not be effective in each and every subject treated, or a majority of treated subjects in a given group or population. An effective amount or a sufficient amount means effectiveness or sufficiency in a particular subject, not a group or the general population. As is typical for such methods, some subjects will exhibit a greater response, or less or no response to a given treatment method or use. Thus, appropriate amounts will depend upon the condition treated, the therapeutic effect desired, as well as the individual subject (e.g., the bioavailability within the subject, gender, age, etc.).

With regard to a disease or symptom thereof, or an underlying cellular response, a detectable or measurable improvement includes a subjective or objective decrease, reduction, inhibition, suppression, limit or control in the occurrence, frequency, severity, progression, or duration of the disease, or complication caused by or associated with the disease, or an improvement in a symptom or an underlying cause or a consequence of the disease, or a reversal of the disease or disorder.

Thus, a successful treatment outcome can lead to a “therapeutic effect,” or “benefit” of decreasing, reducing, inhibiting, suppressing, limiting, controlling or preventing the occurrence, frequency, severity, progression, or duration of a disease or disorder, or one or more adverse symptoms or underlying causes or consequences of the disease in a subject. Treatment methods and uses affecting one or more underlying causes of the disease or adverse symptoms are therefore considered to be beneficial. A decrease or reduction in worsening, such as stabilizing the disease, or an adverse symptom thereof, is also a successful treatment outcome.

Disclosed methods and uses can be combined with any compound, agent, drug, treatment or other therapeutic regimen or protocol having a desired therapeutic, beneficial, additive, synergistic or complementary activity or effect. Exemplary combination compositions and treatments include second actives, such as, biologics (proteins), agents and drugs. Such biologics (proteins), agents, drugs, treatments and therapies can be administered or performed prior to, substantially contemporaneously with or following any other method or use of the disclosure.

The compound, agent, drug, treatment or other therapeutic regimen or protocol can be administered as a combination composition, or administered separately, such as concurrently or in series or sequentially (prior to or following) delivery or administration of a subject LNP as described herein. The disclosure therefore provides combinations where a method or use of the disclosure is in a combination with any compound, agent, drug, therapeutic regimen, treatment protocol, process, remedy or composition, set forth herein or known to one of skill in the art. The compound, agent, drug, therapeutic regimen, treatment protocol, process, remedy or composition can be administered or performed prior to, substantially contemporaneously with or following administration of an LNP as described herein, to a subject.

### Kits

Provided are kits/systems for carrying out a subject method. Such kits comprise various combinations of components useful in any of the methods described elsewhere herein.

A kit can further include one or more additional reagents, where such additional reagents can be any convenient reagent. Components of a subject kit can be in separate containers; or can be combined in a single container. In some cases one or more of a kit's components are pharmaceutically formulated for administration to a human.

In addition to above-mentioned components, a subject kit can further include instructions for using the components of the kit to practice the subject methods (e.g., dosing instructions, instructions to administer the component(s) to an individual. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In some embodiments, the instructions are present as an electronic storage data file present on a suitable computer readable storage medium, e.g. CD-ROM, diskette, flash drive, etc. In some embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Exemplary Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of ordinary skill in the art upon reading this disclosure, each of the individually numbered aspects may be used or combined with any of the preceding or following individually numbered aspects. This is intended to provide support for all such combinations of aspects and is not limited to combinations of aspects explicitly provided below. It will be apparent to one of ordinary skill in the art that various changes and modifications can be made without departing from the spirit or scope of the invention.
1. A composition for modifying a target nucleic acid, comprising a lipid nanoparticle (LNP) that comprises:
   (a) an mRNA encoding a targetable nuclease;
   (b) an mRNA encoding a DNA-binding protein; and
   (c) a donor template comprising:
      (i) a homology directed repair (HDR) template comprising a nucleic acid sequence for insertion into the target nucleic acid; and
      (ii) one or more DNA-binding protein target sequences.
2. The composition of 1, wherein the donor template comprises single stranded DNA (ssDNA).
3. The composition of 1, wherein the donor template comprises double stranded DNA (dsDNA).
4. The composition of any of 1 to 3, wherein the donor template comprises one or more DNA-binding protein target sequences in the region 5′ of the HDR template.
5. The composition of any of 1 to 3, wherein the donor template comprises one or more DNA-binding protein target sequences in the region 3′ of the HDR template.
6. The composition of any of 1 to 3, wherein the donor template comprises one or more DNA-binding protein target sequences in the region 5′ of the HDR template and one or more DNA-binding protein target sequences in the region 3′ of the HDR template.
7. The composition of any of 1 to 6, wherein the targetable nuclease is fused to one or more nuclear localization signal (NLS) sequences.
8. The composition of any of 1 to 7, wherein the LNP comprises one or more ionizable lipids.
9. The composition of 8, wherein the one or more ionizable lipids include: OF-02, L-319, BP lipid 312, LP01, Lipid III-45, ALC-0315, lipid A9, D-Lin, DLin-MC3-DMA, ALC-0315, SM-102, LP01, CL1, TCL053, CKK-E12, ATX-002, DLin-DMA, DLenDMA, DLin-D-DMA, DLin-K-DMA, DLin-M-C2-DMA, DLin-K-DMA, DLin-KC2-DMA, DLin-KC3-DMA, DLin-KC4-DMA, DLin-C2K-DMA, DLin-MP-DMA, DODMA, 98N12-5, C12-200, DLin-C-DAP, DLin-DAC, DLinDAP, DLinAP, DLin-EG-DMA, DLin-2-DMAP, KL10, KL22, KL25, Octyl-CLinDMA, Octyl-CLinDMA (2R), Octyl-CLinDMA (2S), or an analog thereof, or any combination thereof.
10. The composition of any of 1 to 9, wherein the LNP comprises: OF-02, DOPE, cholesterol, DMG-PEG 2000, or DOTAP, or any combination thereof.
11. The composition of any of 1 to 9, wherein the LNP comprises: L-319, DOPE, cholesterol, or DMG-PEG 2000, or any combination thereof.
12. The composition of any of 1 to 11, wherein the total lipid to nucleic acid weight ratio is in a range of from 40:1 to 1:40.
13. The composition of any of 1 to 12 wherein the targetable nuclease is a first RNA-guided nuclease and the DNA-binding protein is a second RNA-guided nuclease.
14. The composition of any of 1 to 12, wherein one RNA-guided nuclease is both the targetable nuclease and the DNA-binding protein.
15. The composition of 14, wherein the RNA-guided nuclease is a CRISPR-Cas effector protein.
16. The composition of any of 13 to 15, wherein the composition further comprises a target guide RNA (gRNA) and a donor gRNA; wherein the donor template further comprises one or more protospacer adjacent motifs (PAMs); wherein the target gRNA is complementary to the target nucleic acid; and wherein at least one of said one or more DNA-binding protein target sequences hybridizes to the donor gRNA's guide sequence or a portion thereof.
17. The composition of 16, wherein one guide RNA is both the target gRNA and the donor gRNA, and wherein said at least one DNA-binding protein target sequence hybridizes to a portion of said guide RNA's guide sequence.
18. The composition of 16, wherein the target gRNA and the donor gRNA have different sequences.
19. The composition of any of 16 to 18, wherein at least one of the one or more DNA-binding protein target sequences is complementary to an equal length portion of the sequence of the donor gRNA.
20. The composition of any of 1 to 19, wherein at least one of the one or more DNA-binding protein target sequences is a truncated CRISPR-Cas targeting sequence (tCTS).
21. The composition of 20, wherein a PAM is located at the 3′ terminus of each tCTS.
22. The composition of any of 20 to 21, wherein the donor template comprises two tCTSs and two PAMs.
23. The composition of 22, wherein the donor template is a dsDNA and wherein each strand of the donor template comprises one or more tCTSs in the region 5′ of the HDR template.
24. The composition of any of 1 to 12, wherein the DNA-binding protein comprises a transcription activator-like effector (TALE) DNA-binding protein or a zinc finger (ZF) DNA-binding protein.
25. The composition of any of 1 to 12 or 24, wherein the targetable nuclease comprises a transcription activator-like effector nuclease (TALEN).
26. The composition of any of 1 to 12 or 24, wherein the targetable nuclease comprises a zinc finger nuclease (ZFN).
27. A composition for modifying a target nucleic acid, comprising a lipid nanoparticle (LNP) that comprises:
   (a) an mRNA encoding a CRISPR-Cas effector protein;
   (b) a guide RNA (gRNA) comprising a guide sequence that is complementary to the target nucleic acid; and
   (c) a donor template comprising:
      (i) a homology directed repair (HDR) template comprising a nucleic acid sequence for insertion into the target nucleic acid;
      (ii) one or more tCTSs that hybridize to a portion of the guide sequence of the gRNA; and
      (iii) a PAM sequence adjacent to each tCTS.
28. The composition of 27, wherein the donor template comprises one or more tCTSs in the region 5′ of the HDR template, or one or more tCTSs in the region 3′ of the HDR template.
29. The composition of 27, wherein the donor template comprises one or more tCTSs in the region 5′ of the HDR template and one or more tCTSs in the region 3′ of the HDR template.
30. A composition for modifying a target nucleic acid, comprising a lipid nanoparticle (LNP) that comprises:
   (a) an ionizable lipid selected from OF-02 or L-319;
   (b) a targetable nuclease or an mRNA encoding the targetable nuclease;
   (c) a DNA-binding protein or an mRNA encoding the DNA-binding protein; and
   (d) a donor template comprising:
      (i) a homology directed repair (HDR) template comprising a nucleic acid sequence for insertion into the target nucleic acid; and
      (ii) one or more DNA-binding protein target sequences.
31. The composition of 30, wherein the LNP comprises: OF-02, L-319, DOPE, cholesterol, DMG-PEG 2000, or DOTAP, or any combination thereof.
32. The composition of any of 30 to 31, wherein the LNP comprises OF-02.
33. The composition of 32, wherein the LNP comprises 15 mol % to 25 mol % OF-02, 20 mol % to 30 mol % DOPE, 35 mol % to 45 mol % cholesterol, 0.5 mol % to 1.5 mol % DMG-PEG 2000, and 15 mol % to 25 mol % DOTAP of the total lipids.
34. The composition of any of 30 to 31, wherein the LNP comprises L-319.
35. The composition of 34, wherein the LNP comprises 30 mol % to 40 mol % L-319, 20 mol % to 30 mol % DOPE, 35 mol % to 45 mol % cholesterol, and 0.5 mol % to 1.5 mol % DMG-PEG 2000 of the total lipids.
36. The composition of any of 30 to 35, wherein the total lipid to nucleic acid weight ratio is in a range of from 40:1 to 1:40.
37. The composition of any of 30 to 36, wherein the donor template comprises single stranded DNA (ssDNA).
38. The composition of any of 30 to 37, wherein the donor template comprises double stranded DNA (dsDNA).
39. The composition of any of 30 to 38, wherein the donor template comprises one or more DNA-binding protein target sequences in the region 5′ of the HDR template.
40. The composition of any of 30 to 38, wherein the donor template comprises one or more DNA-binding protein target sequences in the region 3′ of the HDR template.
41. The composition of any of 30 to 38, wherein the donor template comprises one or more DNA-binding protein target sequences in the region 5′ of the HDR template and one or more DNA-binding protein target sequences in the region 3′ of the HDR template.
42. The composition of any of 30 to 41, comprising the targetable nuclease.
43. The composition of any of 30 to 41, comprising the mRNA encoding the targetable nuclease.
44. The composition of any of 30 to 43, comprising the DNA-binding protein.
45. The composition of any of 30 to 43, comprising the mRNA encoding the DNA-binding protein.
46. The composition of any of 30 to 45, wherein the targetable nuclease is fused to one or more nuclear localization signal (NLS) sequences.
47. The composition of any of 30 to 46, wherein:
   (a) each of the targetable nuclease and the DNA-binding protein is an RNA-guided nuclease;
   (b) the composition comprises a target guide RNA (gRNA) and a donor gRNA;
   (c) the donor template comprises one or more protospacer adjacent motifs (PAMs);
   (d) the target gRNA is complementary to the target nucleic acid.
48. The composition of 47, wherein one RNA-guided nuclease is both the targetable nuclease and the DNA-binding protein, and the one or more DNA-binding protein target sequences are truncated CRISPR-Cas targeting sequences (tCTS) that hybridize to the donor gRNA's guide sequence or a portion thereof.
49. The composition of any of 47 to 48, wherein the RNA-guided nuclease is a CRISPR-Cas effector protein.
50. The composition of any of 47 to 49, wherein one guide RNA is both the target gRNA and the donor gRNA, and wherein said at least one DNA-binding protein target sequence hybridizes to a portion of said guide RNA's guide sequence.
51. The composition of any of 49 to 50, wherein the CRISPR-Cas effector protein is complexed with the target gRNA or the donor gRNA as a ribonucleoprotein (RNP).
52. The composition of 51, wherein the RNP comprising the donor gRNA is complexed with the donor template.
53. The composition of any of 47 to 52, wherein a PAM is located at the 3′ terminus of each tCTS.
54. The composition of any of 47 to 53, wherein the donor template comprises two tCTSs and two PAMs.
55. The composition of 54, wherein the donor template comprises one or more tCTSs in the region 5′ of the HDR template and comprises one or more tCTSs in the region 3′ of the HDR template.
56. The composition of any of 30 to 46, wherein the DNA-binding protein comprises a transcription activator-like effector (TALE) DNA-binding protein or a zinc finger (ZF) DNA-binding protein.
57. The composition of any of 30 to 46 or 56, wherein the targetable nuclease comprises a transcription activator-like effector nuclease (TALEN).
58. The composition of any of 30 to 46 or 56, wherein the targetable nuclease comprises a zinc finger nuclease (ZFN).
59. A composition for modifying a target nucleic acid, comprising a lipid nanoparticle (LNP) that comprises:
   (a) an ionizable lipid selected from OF-02 or L-319;
   (b) a CRISPR-Cas effector protein or an mRNA encoding the CRISPR-Cas effector protein;
   (c) a gRNA that is complementary to the target nucleic acid; and
   (d) a donor template comprising:
      (i) a homology directed repair (HDR) template comprising a nucleic acid sequence for insertion into the target nucleic acid; and
      (ii) one or more tCTSs that hybridize to a portion of the gRNA's guide sequence; and
      (iii) a PAM sequence adjacent to each tCTS.
60. The composition of 59, wherein the LNP comprises 15 mol % to 25 mol % OF-02, 20 mol % to 30 mol % DOPE, 35 mol % to 45 mol % cholesterol, 0.5 mol % to 1.5 mol % DMG-PEG 2000, and 15 mol % to 25 mol % DOTAP of the total lipids.
61. The composition of 59, wherein the LNP comprises 30 mol % to 40 mol % L-319, 20 mol % to 30 mol % DOPE, 35 mol % to 45 mol % cholesterol, and 0.5 mol % to 1.5 mol % DMG-PEG 2000 of the total lipids.
62. The composition of any of 59 to 61, wherein the donor template comprises one or more tCTSs in the region 5′ of the HDR template, or one or more tCTSs in the region 3′ of the HDR template.
63. The composition of any of 59 to 61, wherein the donor template comprises one or more tCTSs in the region 5′ of the HDR template and comprises one or more tCTSs in the region 3′ of the HDR template.
64. A method for modifying a target nucleic acid in a cell, comprising contacting a cell with the composition of any of 1 to 63, wherein said nucleic acid sequence for insertion is integrated into the target nucleic acid.
65. The method of 64, wherein the method is performed in vivo, in vitro, or ex vivo.
66. The method of any of 64 to 65, wherein the introducing comprises electroporation.
67. The method of any of 64 to 66, wherein the cell is a human cell.
68. The method of any of 64 to 67, wherein the cell is a primary cell.
69. The method of any of 64 to 68, wherein the cell is a T cell.
70. The method of 69, wherein said nucleic acid sequence for insertion is integrated into the target nucleic acid at a T-cell receptor a constant (TRAC) locus.
71. The method of any of 69 to 70, wherein the HDR template encodes a chimeric antigen receptor (CAR).
72. The method of any of 64 to 68, wherein the cell is a hematopoietic stem cell (HSC).
73. The method of any of 64 to 66, wherein the cell is a HEK293T cell.
74. The method of any of 64 to 72, wherein said contacting results in treatment of a subject.
75. The method of 74, wherein the target nucleic acid in the cell is modified ex vivo and the cell is introduced into the subject.
76. The method of 74, wherein the target nucleic acid in the cell is modified in vivo.
77. The method of any of 74 to 76, wherein the subject is an animal.
78. The method of 77, wherein the subject is a mammal.
79. The method of 78, wherein the subject is a human.

### EXPERIMENTAL EXAMPLES

The following examples are provided for purposes of illustration only, and are not intended to be limiting unless otherwise specified. Thus, the invention should in no way be construed as being limited to the following examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like

### Example 1: Application of Truncated CRISPR-Cas Targeting (e.g., Truncated CRISPR-Cas9 Targeting) Sequence (tCTS) for Lipid Nanoparticle (LNP) Delivery [Example Embodiment]

Lipid nanoparticles (LNPs) are loaded with tCTS-modified DNA HDR template, Cas9 mRNA (or Cas9 protein), and sgRNA. The LNP is then delivered to primary human T-cells using a LNP formulation containing a mixture of ionizable and excipient lipids (e.g., OF-02, DOPE, cholesterol, DMG-PEG 2000). Upon intracellular delivery of the genome editing cargo, Cas9 mRNA is translated to an endonuclease protein, which binds the sgRNA and forms a ribonucleoprotein (RNP) complex. The tCTS then mediates high-specificity, reversible binding of the RNP to the tCTS-modified DNA HDR template in situ. This facilitates active nuclear transport and efficient homology-directed repair (HDR). The RNP-DNA complex is then rapidly shuttled into the cell nuclei leveraging the nuclear localization signal (NLS) of the Cas9 protein, though other proteins with NLS could be substituted. A schematic representation of LNP-mediated delivery for efficient HDR in human T-cells is shown in FIG. 1. Although the figure depicts addition of tCTS via PCR, a tCTS can be added to a donor template using any convenient method, e.g., using any convenient molecular biology technique (e.g., cloned directly into a plasmid).

### Example 2: LNP-Mediated Large Gene (Rab11-sfGFP) Knock-In to the Rab11 Locus in Primary Human T-Cells

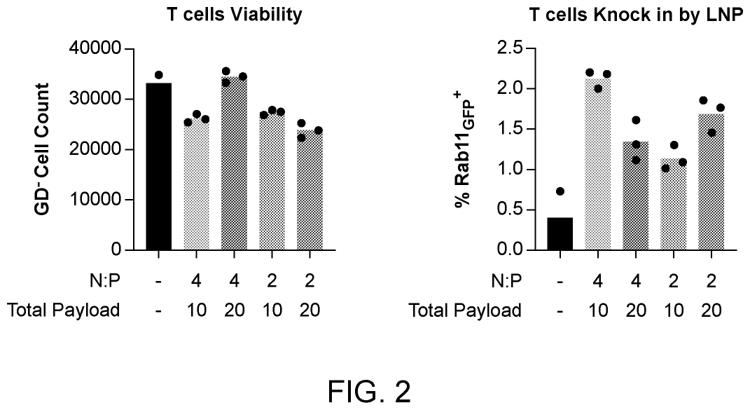

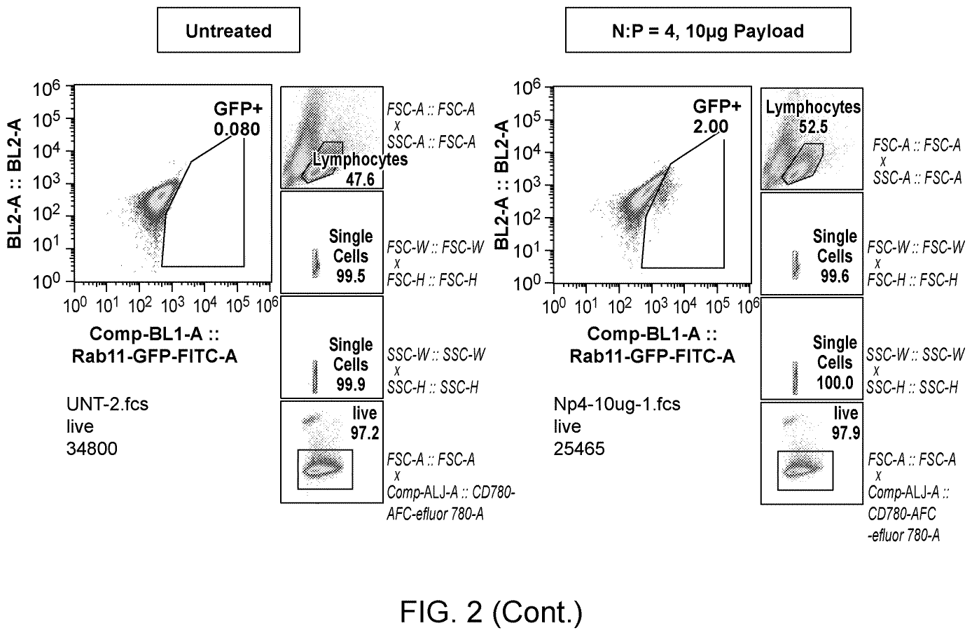

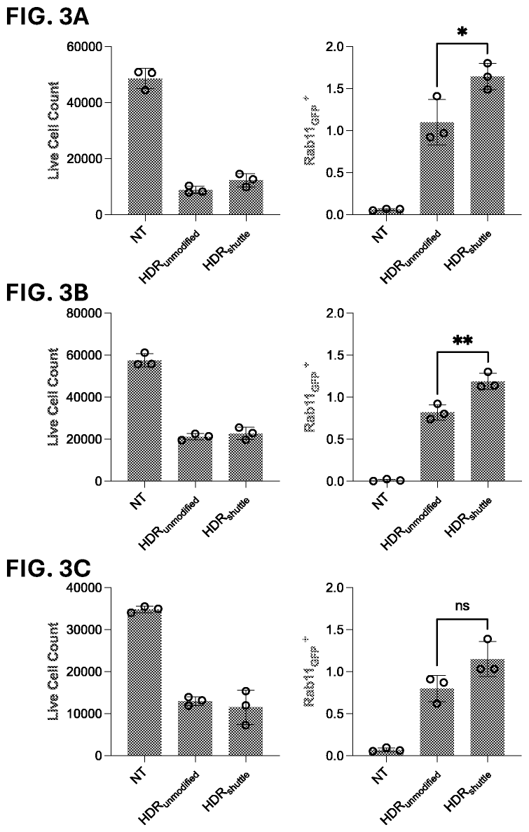

As shown in FIG. 2, Rab11 sgRNA, Cas9 mRNA, and tCTS-tagged Rab11-sfGFP DNA template were encapsulated in a single lipid nanoparticle (LNP) at different amine-to-phosphate (N:P) ratios. Activated primary human T cells were treated with LNPs at low (10 μg/million cells) and high (20 μg/million cells) nucleic acid payloads. After 72 hours, human T cells were collected, and flow cytometry was performed. Knock-in efficiency was determined by calculating the percentage of GFP+ cells among live cells using the GFP reporter in the knock-in template.

### Example 3: Truncated Cas9-Targeted Sequences (tCTS) Improve HDR Knock-In Efficiency in Primary Human T Cells

Primary human T-cells were transfected with a single lipid nanoparticle formulation containing Rab11 sgRNA, Cas9 mRNA, and either tCTS-modified (HDRₛₕᵤₜₜₗₑ) or unmodified (HDR_{unmodified}) template (50 μg total/million cells). The HDR template contains sfGFP reporter gene, and the knock-in efficiency was determined by the percentage of sfGFP positive in live cells after 3 (FIG. 3A), 5 (FIG. 3B), and 7 (FIG. 3C) days post-transfection. Statistical analysis was performed using t-tests, with significance set at p<0.05.

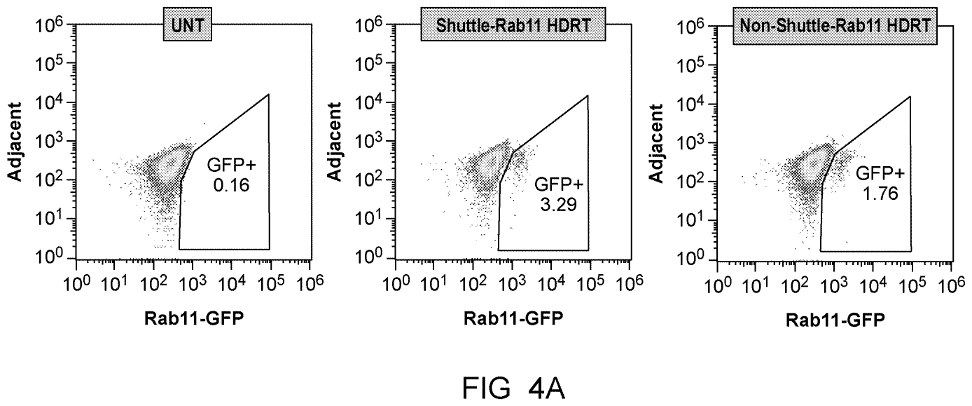

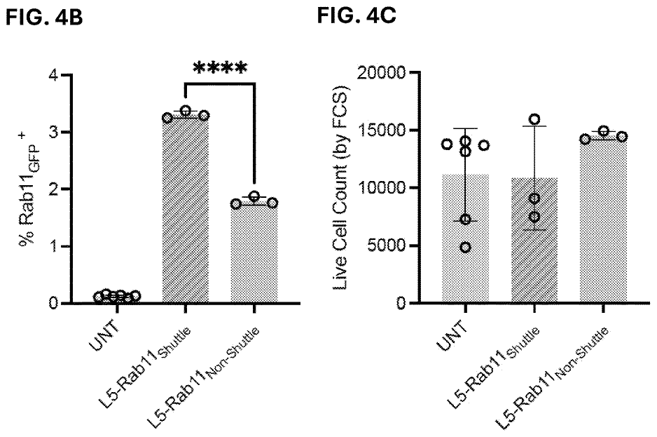

In addition, primary human T-cells were transfected with a single lipid nanoparticle formulation containing Rab11 sgRNA, Cas9 mRNA, and either tCTS-modified (Rab11_{Shuttle}) or unmodified (Rab11_{Non-Shuttle}) template (50 μg total cargoes/10⁶ cells). The HDR template contains sfGFP reporter gene, and the knock-in efficiency was determined by the percentage of sfGFP positive in live cells after 3 days post-transfection. Statistical analysis was performed using t-tests. (****, p<0.0001), and the results are shown in FIG. 4A-C.

### Example 4: Methods for RNP-HDRT Delivery to HEK293T Cells

Ribonucleoprotein complex (RNP) encapsulation into lipid nanoparticles (LNPs) was achieved using microfluidic mixing via the NanoAssemblr Spark (Precision NanoSystems, Marlborough, MA). The aqueous phase, containing the RNP, was prepared by combining Rab11 single guide RNA (sgRNA) and Cas9 protein (QB3 MacroLabs, UC Berkeley) in nuclease-free water at final concentrations of 28.57 μM and 14.28 μM, respectively. The mixture was incubated at 37° C. for 15 minutes to facilitate complex formation. Subsequently, a homology-directed repair (HDR) DNA template encoding superfold GFP (sfGFP), flanked by Rab11 homology arms with guide-specific truncated Cas9 target sequences (tCTS) at each end, was added at a final concentration of 0.26 μg/μL. The solution was incubated at room temperature for 5 minutes to enable the HDRT to complex with the RNP. This complex was delivered via a singular LNP.

The organic phase was prepared by dissolving lipid components in ethanol, consisting of OF-02 (https://www.caymanchem.com/product/37652/of-02) (18.33 mol %), DOPE (24.28 mol %), cholesterol (38.94 mol %), DMG-PEG 2000 (1.2 mol %), and DOTAP (17.24 mol %). For RNP-LNP formation, 19 μL of the prepared aqueous phase was further diluted with 13 μL of nuclease-free water and mixed with 16 μL of the organic lipid phase using the preset program (#3) on the NanoAssemblr Spark. The resulting LNP suspension was dialyzed against calcium- and magnesium-free Dulbecco's phosphate-buffered saline (DPBS) for 1 hour before use in cell treatment experiments.

HEK293T cells were seeded at 80,000 cells per well in a 24-well plate in 500 μL of DMEM+10% FBS and left to adhere overnight. The cells were treated with 50 pmol of dialyzed LNP with RNP/HDRT encapsulated in it. After 3 days of treatment, HEK293T cells were collected for flow cytometry. Cells were lifted with 200 μL of 0.5 mM EDTA and 100 μL was taken for flow cytometry.

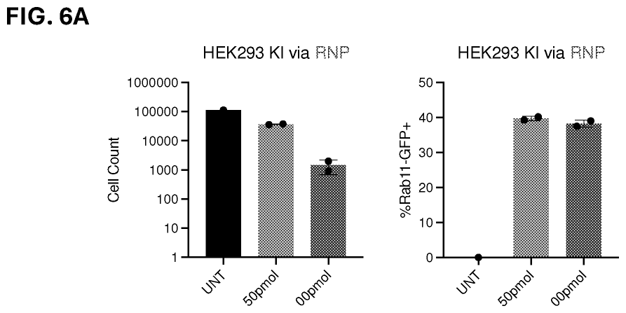

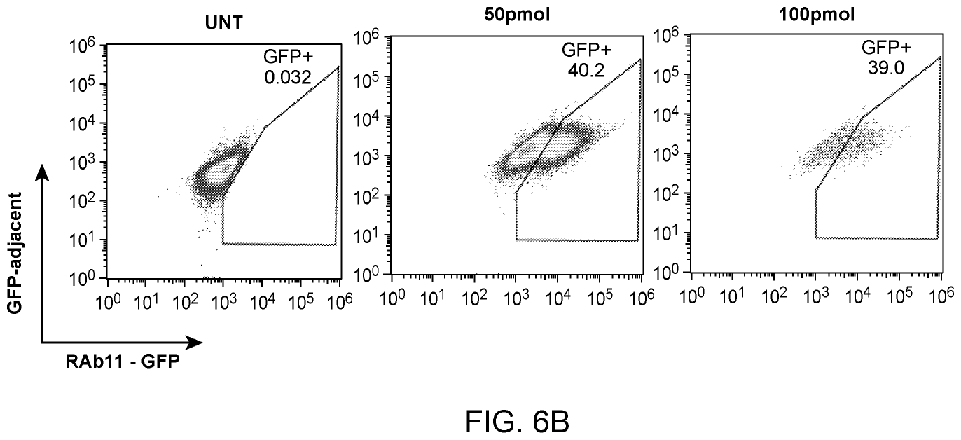

FIGS. 6A and 6B show the results of LNP-mediated HDR knock-in to HEK293T cells with donor DNA-sgRNA-Cas9 ribonucleoprotein (RNP) complex. LNP was formulated by adding lipid mixture, containing OF-02 (18.33 mol %), DOPE (24.28 mol %), cholesterol (38.94 mol %), DMG-PEG 2000 (1.2 mol %) and DOTAP (17.24 mol %) to pre-formed sgRNA, Cas9 protein, and donor DNA (mol:mol:mol) RNP complex. RNP was formulated at 50 and 100 pmol with polyglutamic acid (PGA) at a ratio of 0.8 PGA:1 sgRNA. The donor DNA template has guide-specific truncated Cas9 target sequences (tCTS) at each end. Encapsulation into LNP was performed by Microfluidic mixing (NanoAssemblr Spark). HEK293 cells were treated with an equivalence of 50 pmol and 100 pmol. Successful homology-directed repair was determined by sfGFP expression via flow cytometry from the donor DNA template designed to integrate into Rab11 locus following cutting. Up to 40% of HEK293 cells were successfully knocked-in after 3 days post-treatment seen on flow cytometry. FIG. 6B shows representative flow cytometry plots of HEK293 cells treated with RNP-LNP, indicating successful knock-in of sfGFP into Rab11 locus.

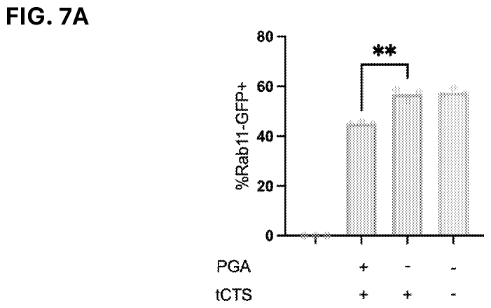

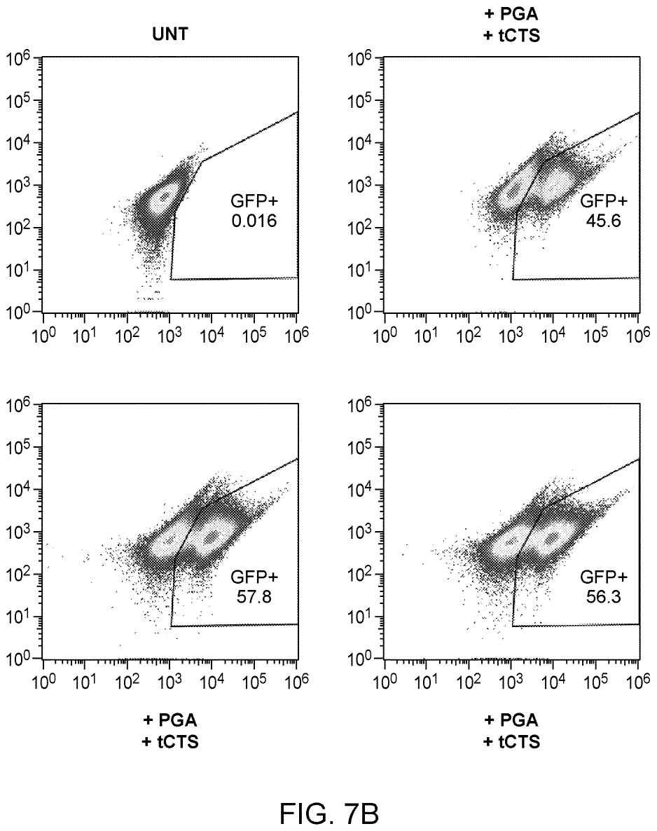

FIGS. 7A and 7B show the results of LNP-mediated HDR knock-in to HEK293T cells with donor DNA-sgRNA-Cas9 ribonucleoprotein (RNP) complex. LNP was formulated by adding lipid mixture, containing OF-02 (18.33 mol %), DOPE (24.28 mol %), cholesterol (38.94 mol %), DMG-PEG 2000 (1.2 mol %) and DOTAP 17.24 mol %) to pre-formed sgRNA, Cas9 protein, and donor DNA (mol:mol:mol) RNP complex. RNP was made at 50 pmol with and without polyglutamic acid (PGA). PGA was added at a ratio of 0.8 PGA:1 sgRNA. Donor DNA template with and without guide-specific truncated Cas9 target sequences (tCTS) at each end were tested. Encapsulation into LNP was performed by microfluidic mixing (NanoAssemblr Spark). Successful homology-directed repair was determined by sfGFP expression from the donor DNA template designed to integrate into Rab11 locus following the cutting. As determined by the flow cytometry, up to 60% of HEK293 cells were successfully knocked-in after 3 days post-treatment. FIG. 7B shows representative flow cytometry plots of HEK293 cells treated with RNP-LNP, indicating successful knock-in of sfGFP into Rab11 locus.

### Example 5: Methods for sqRNA+Cas9 mRNA-HDRT Delivery to Primary Human T-Cells

Preparation of mRNA-LNP for Rab11 sgRNA, Cas9 mRNA, and Rab11-GFP DNA donor templates was carried out using ethanol injection. Briefly, Rab11 sgRNA, Cas9 mRNA, and Rab11-GFP HDR donor templates (for inserting sfGFP into the Rab11 locus) were diluted in a pH 4 aqueous buffer to final concentrations of 0.068 μg/μL, 0.068 μg/μL, and 0.034 μg/μL, respectively. An organic lipid mixture containing L-319 (caymanchem product 35051/1-319) (35.55% mol), DOPE (24.29% mol), Cholesterol (38.95% mol), and DMG-PEG2000k (1.2% mol) was then pipette-mixed with the aqueous phase at a 1:3 v/v ratio. The resulting LNP is ready for subsequent cell experiments.

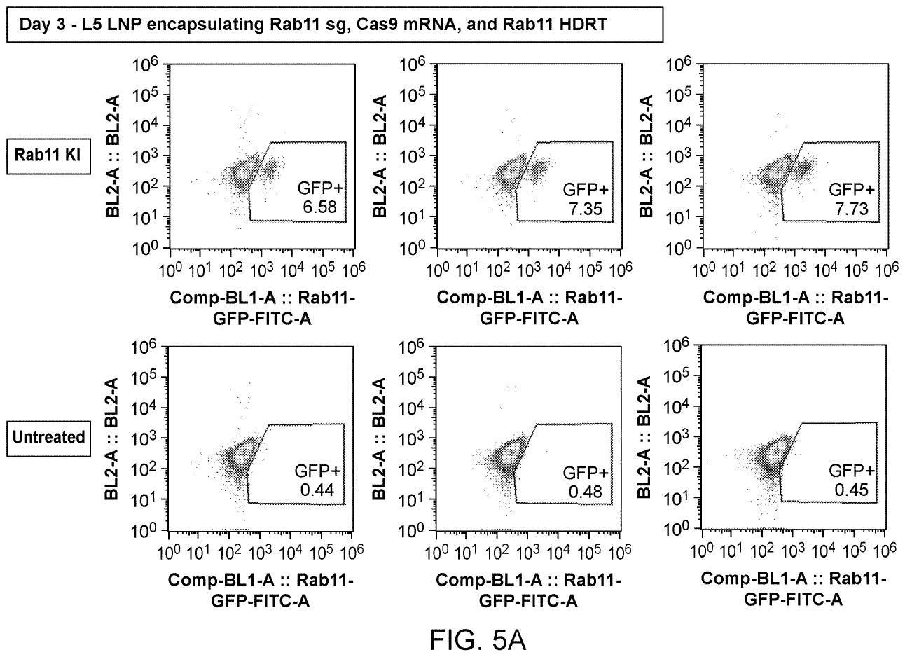

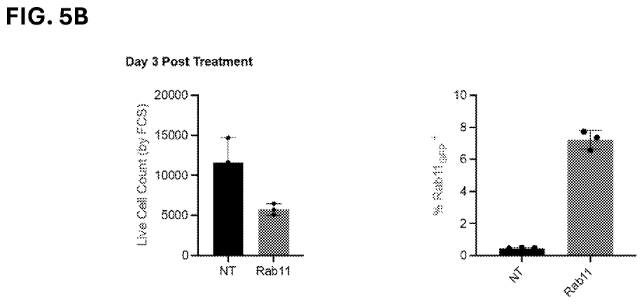

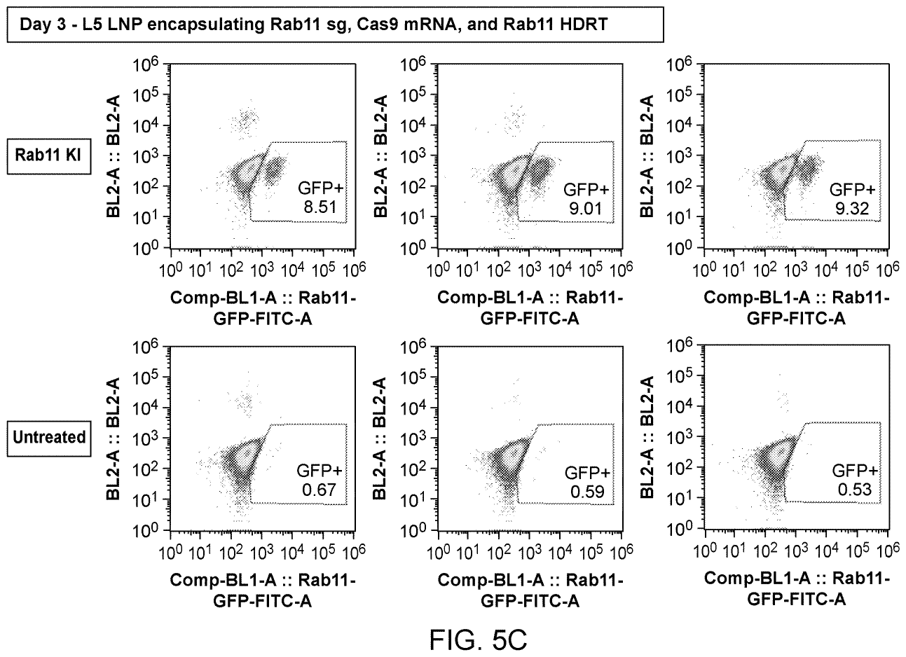

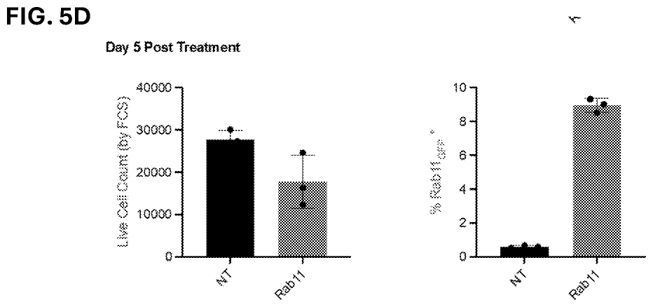

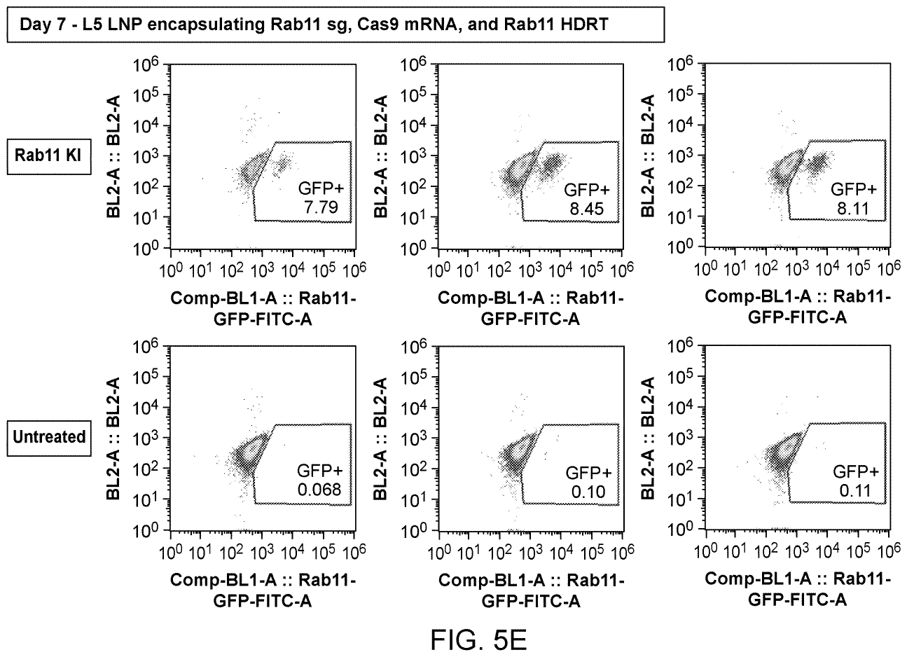

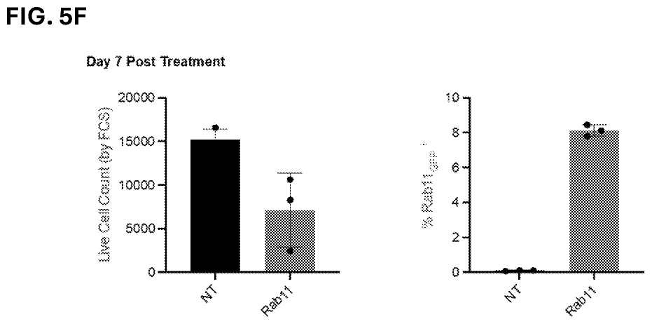

Primary human T-cells were isolated from a fresh leukopak and activated with 300 U/mL IL-2, 5 ng/mL of IL-7, 5 ng/mL of IL-15, and a 1:1 ratio of CD3/CD28 Dynabeads:Cells. After 48 hours, cells were de-beaded via magnetic separation and seeded at 50,000 cells per well in a 96-well round bottom plate in 100 μL of X-VIVIO 15 media supplemented with 500 U/mL IL-2. LNP treatment was added on top of cells at 10 μg per million cells in 100 μL X-VIVO 15 media+500 U/mL IL-2+2 μg/mL ApoE, for a final concentration of 1 μg/mL ApoE after treatment. 0% FBS was used during treatment. Media was changed on day 3 with 500 U/mL IL-2 and again on day 5 with 12 uL of Immunocult and 500 U/mL IL-2. After 3, 5, or 7 days of treatment, T-cells are collected for flow cytometry. Results are shown in FIGS. 5A and 5B (day 3), FIGS. 5C and 5D (day 5), and FIGS. 5E and 5F (day 7).

### Example 6: LNP Delivery of Single Stranded DNA Templates for Homology-Directed Repair with Dual Cas9 Shuttles

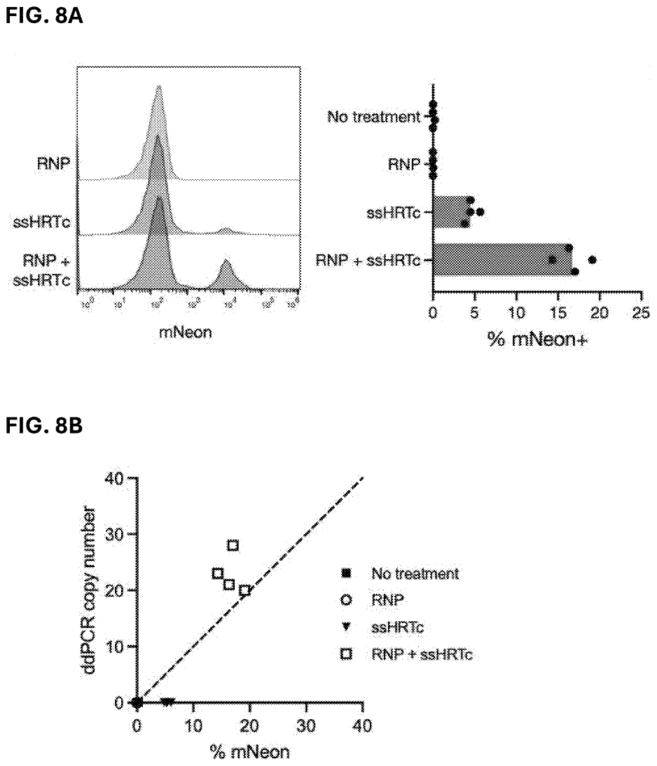

As lipid nanoparticles (LNP) and Cas9 RNP both have a net positive charge, LNP can be used to deliver ssDNA templates for homology directed repair, as the ssDNA is negatively charged. Here we tested if LNPs could be used to deliver long ssDNA templates for homology directed repair. Moreover, ssDNA templates would 1) already carry tCTSs on the ends of the template (and thereby interact with Cas9) and 2) promote co-packaging with Cas9. The Cas9 molecules were engineered with nuclear localization signals, allowing the template to be shuttled to the nucleus after endosomal escape. ssDNA template is half the molecular weight of dsDNA, allowing for longer templates to be packaged within LNP. LNP delivery of a long single strand DNA template (plus small oligos to hybridize to the tCTSs on the ends in order to be double stranded to allow Cas9 to bind) encoding EF1a-mNeon and Cas9 with gRNA targeting AAVS1 HEK293T cells was tested, and durable targeted integration was observed 20 days after delivery (FIG. 8) as measured by mNeon expression and in-out digital droplet PCR. Template alone resulted in minor background mNeon expression (FIG. 8A: representative flow cytometry plot of mNeon in HEK293T cells 20 days after lipid nanoparticle administration), which was likely episomal expression from the ~10% contaminating dsDNA in lab-made templates, as there was no evidence of targeted integration by ddPCR for the template alone (FIG. 8B: ddPCR copy number analysis by in-out PCR for knock-in at AAVS1 compared to mNeon expression).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1-7. (canceled)

8. The composition of claim 27, wherein the LNP comprises one or more ionizable lipids.

9. The composition of claim 8, wherein the one or more ionizable lipids include: OF-02, L-319, BP lipid 312, LP01, Lipid 111-45, ALC-0315, lipid A9, D-Lin, DLin-MC3-DMA, ALC-0315, SM-102, LP01, CL1, TCL053, CKK-E12, ATX-002, DLin-DMA, DLenDMA, DLin-D-DMA, DLin-K-DMA, DLin-M-C2-DMA, DLin-K-DMA, DLin-KC2-DMA, DLin-KC3-DMA, DLin-KC4-DMA, DLin-C2K-DMA, DLin-MP-DMA, DODMA, 98N12-5, C12-200, DLin-C-DAP, DLin-DAC, DLinDAP, DLinAP, DLin-EG-DMA, DLin-2-DMAP, KL10, KL22, KL25, Octyl-CLinDMA, Octyl-CLinDMA (2R), Octyl-CLinDMA (2S), or an analog thereof, or any combination thereof.

10. The composition of claim 27, wherein the LNP comprises:
OF-02, DOPE, cholesterol, DMG-PEG 2000, or DOTAP, or any combination thereof.

11. The composition of claim 27, wherein the LNP comprises:
L-319, DOPE, cholesterol, or DMG-PEG 2000, or any combination thereof.

12. The composition of claim 27, wherein the total lipid to nucleic acid weight ratio of the LNP is in a range of from 40:1 to 1:40.

13-26. (canceled)

27. A composition for modifying a target nucleic acid, comprising a lipid nanoparticle (LNP) that comprises:
(a) an mRNA encoding a CRISPR-Cas effector protein;
(b) a guide RNA (gRNA) comprising a guide sequence that is complementary to the target nucleic acid; and
(c) a donor template comprising:
(i) a homology directed repair (HDR) template comprising a nucleic acid sequence for insertion into the target nucleic acid;
(ii) one or more tCTSs that hybridize to a portion of the guide sequence of the gRNA; and
(iii) a PAM sequence adjacent to each tCTS.

28. The composition of claim 27, wherein the donor template comprises one or more tCTSs in the region 5′ of the HDR template, and/or one or more tCTSs in the region 3′ of the HDR template.

29-30. (canceled)

31. The composition of claim 59, wherein the LNP comprises: DOPE, cholesterol, DMG-PEG 2000, or DOTAP, or any combination thereof.

32. The composition of claim 59, wherein the LNP comprises OF-02.

33. (canceled)

34. The composition of claim 59, wherein the LNP comprises L-319.

35. (canceled)

36. The composition of claim 59, wherein the total lipid to nucleic acid weight ratio of the LNP is in a range of from 40:1 to 1:40.

37-45. (canceled)

46. The composition of claim 59, wherein the CRISPR-Cas effector protein is fused to one or more nuclear localization signal (NLS) sequences.

47-50. (canceled)

51. The composition of claim 59, wherein the CRISPR-Cas effector protein is complexed with the target gRNA or the donor gRNA as a ribonucleoprotein (RNP).

52. The composition of claim 51, wherein the RNP comprising the donor gRNA is complexed with the donor template.

53-58. (canceled)

59. A composition for modifying a target nucleic acid, comprising a lipid nanoparticle (LNP) that comprises:
(a) an ionizable lipid selected from OF-02 or L-319;
(b) a CRISPR-Cas effector protein or an mRNA encoding the CRISPR-Cas effector protein;
(c) a gRNA that is complementary to the target nucleic acid; and
(d) a donor template comprising:
(i) a homology directed repair (HDR) template comprising a nucleic acid sequence for insertion into the target nucleic acid; and
(ii) one or more truncated CRISPR-Cas targeting sequences tCTSs that hybridize to a portion of the gRNA's guide sequence; and
(iii) a PAM sequence adjacent to each tCTS.

60. The composition of claim 59, wherein the LNP comprises 15 mol % to 25 mol % OF-02, 20 mol % to 30 mol % DOPE, 35 mol % to 45 mol % cholesterol, 0.5 mol % to 1.5 mol % DMG-PEG 2000, and 15 mol % to 25 mol % DOTAP of the total lipids.

61. The composition of claim 59, wherein the LNP comprises 30 mol % to 40 mol % L-319, 20 mol % to 30 mol % DOPE, 35 mol % to 45 mol % cholesterol, and 0.5 mol % to 1.5 mol % DMG-PEG 2000 of the total lipids.

62. The composition of claim 59, wherein the donor template comprises one or more tCTSs in the region 5′ of the HDR template, and/or one or more tCTSs in the region 3′ of the HDR template.

63. (canceled)

64. A method for modifying a target nucleic acid in a cell, comprising contacting a cell with the composition of claim 27, wherein said nucleic acid sequence for insertion is integrated into the target nucleic acid.

65-68. (canceled)

69. The method of claim 64, wherein the cell is a T cell, and optionally wherein the nucleic acid sequence for insertion encodes a chimeric antigen receptor (CAR) that is integrated into the target nucleic acid at a T-cell receptor a constant (TRAC) locus.

70-79. (canceled)

80. A method for modifying a target nucleic acid in a cell, comprising contacting a cell with the composition of claim 59, wherein said nucleic acid sequence for insertion is integrated into the target nucleic acid.

81. The method of claim 80, wherein the cell is a T cell, and optionally wherein the nucleic acid sequence for insertion encodes a chimeric antigen receptor (CAR) that is integrated into the target nucleic acid at a T-cell receptor a constant (TRAC) locus.


SEQUENCE LISTING (19 sequences)

SEQ ID NO: 1 - protein, 20 aa, synthetic construct
AVKRPAATKKAGQAKKKKLD

SEQ ID NO: 2 - protein, 25 aa, synthetic construct
MSRRRKANPTKLSENAKKLAKEVEN

SEQ ID NO: 3 - protein, 9 aa, synthetic construct
PAAKRVKLD

SEQ ID NO: 4 - protein, 9 aa, synthetic construct
KLKIKRPVK

SEQ ID NO: 5 - protein, 7 aa, synthetic construct
PKKKRKV

SEQ ID NO: 6 - protein, 16 aa, synthetic construct
KRPAATKKAGQAKKKK

SEQ ID NO: 7 - protein, 11 aa, synthetic construct
RQRRNELKRSP

SEQ ID NO: 8 - protein, 38 aa, synthetic construct
NQSSNFGPMKGGNFGGRSSGPYGGGGQYFAKPRNQGGY

SEQ ID NO: 9 - protein, 42 aa, synthetic construct
RMRIZFKNKGKDTAELRRRRVEVSVELRKAKKDEQILKRRNV

SEQ ID NO: 10 - protein, 8 aa, synthetic construct
VSRKRPRP

SEQ ID NO: 11 - protein, 8 aa, synthetic construct
PPKKARED

SEQ ID NO: 12 - protein, 8 aa, synthetic construct
PQPKKKPL

SEQ ID NO: 13 - protein, 12 aa, synthetic construct
SALIKKKKKMAP

SEQ ID NO: 14 - protein, 5 aa, synthetic construct
DRLRR

SEQ ID NO: 15 - protein, 7 aa, synthetic construct
PKQKKRK

SEQ ID NO: 16 - protein, 10 aa, synthetic construct
RKLKKKIKKL

SEQ ID NO: 17 - protein, 10 aa, synthetic construct
REKKKFLKRR

SEQ ID NO: 18 - protein, 20 aa, synthetic construct
KRKGDEVDGVDEVAKKKSKK

SEQ ID NO: 19 - protein, 17 aa, synthetic construct
RKCLQAGMNLEARKTKK